(19) United States
(12) Patent Application Publication
(10) Pub. No.: US 2022/0412955 A1
(43) Pub. Date: Dec. 29, 2022

(54) A MODEL OF CLINICAL SYNERGY IN CANCER

(71) Applicant: H. LEE MOFFITT CANCER CENTER AND RESEARCH INSTITUTE, INC., Tampa, FL (US)

(72) Inventors: Aristo S. SILVA, Tampa, FL (US); Kenneth SHAIN, Saint Petersburg, FL (US); Praneeth SUDALAGUNTA, Tampa, FL (US); Rafael Renatino CANEVAROLO, Tampa, FL (US); Mark MEADS, Tampa, FL (US)

(21) Appl. No.: 17/779,870

(22) PCT Filed: Nov. 25, 2020
(86) PCT No.: PCT/US2020/062232

§ 371 Date: May 25, 2022

Related U.S. Application Data
(60) Provisional application No. 62/940,223, filed on Nov. 25, 2019.

(51) Int. Cl. G01N 33/50; G16B 5/00; G06T 7/20; G06T 7/00

(52) U.S. Cl. CPC G01N 33/5011; G16B 5/00; G06T 7/20; G06T 7/0012; G01N 2500/10; G06T 2207/30096

(57) ABSTRACT

Disclosed is a method of detecting synergistic drug combinations for the treatment of a cancer, comprising: culturing infected cells in a chamber: contacting the cells in with a first active agent; measuring and/or estimating the concentration of the first active agent at a first and second time point; capturing a first and second optical signal from the contacted cells at the first and second time points; analyzing the first optical signal and the second optical signal to detect cell membrane motion of the cells; analyzing the cell membrane motion to quantify the viability of the cells following contact with the first active agent thereby detecting the drug induced damage at the second time point; measuring, calculating, and/or estimating the repair rate of the cells, therapeutic threshold, rate of sensitivity of therapy, and/or clonal composition of the tumor; repeating said steps with a second active agent.

This application claims the benefit of U.S. Provisional Application No. 62/940,223, filed on Nov. 25, 2019 which is incorporated herein by reference in its entirety.

This invention was made with government support under Grant No. CA193489-01A1 awarded by National Institutes of Health. The government has certain rights in the invention.

### I. BACKGROUND

1. Innate or acquired resistance poses a major hurdle in effectively treating many cancers. Resistance to a drug can arise because of enhanced degradation of the drug, increased expression of the drug target, alteration of the target, clonal evolution, microenvironmental factors, or intratumoral heterogeneity. Thus, combination effect can be improved either by combining a drug that disrupts the mechanism of resistance of a second drug, or by combining drugs that target different subpopulations in the tumor. The pursuit for synergistic drug combinations arises from the myriad of advantages of combination therapy, such as maximizing efficacy, reducing toxicity, and addressing interpatient variability, as well as delaying and overcoming innate or acquired resistance.

2. In spite of the advantages seen in combination therapies, there are also adverse drug-drug interactions in patients. Furthermore, a combination proven to be statistically beneficial for a cohort of patients may not be the most promising option for each individual patient in the cohort, some patients can be further benefited by combinations tailored to their particularities and needs. However, absolute personalization of therapy would be impractical based solely on a patient's clinical history and clinical literature. Thus, what is needed are pharmacodynamic methods for identifying therapies that yield better outcomes and complement a physician's clinical acumen.

3. The probability of success (POS) for oncology drugs in a phase III clinical trial is estimated to be 35.5% in comparison to a POS of 63.6% for all other therapeutic groups (endocrinology, cardiovascular, vaccines, etc.). There is a dire need to improve this success rate by preclinically evaluating the efficacy of the experimental arm over a standard-of-care option, especially those involving combination therapies. This motivates the need to employ novel high-throughput combination screening tools preclinically that can improve the POS of phase III clinical trials in oncology.

### II. SUMMARY

4. Disclosed are methods and compositions related to non-destructive methods for identifying combination therapies and treatment regimens.

5. A method detecting synergistic drug combinations for the treatment of a cancer comprising culturing a plurality of cells from a subject in a chamber; contacting the cells in the chamber with a first active agent; measuring and/or estimating the concentration of the first active agent at a first time point; capturing a first optical signal from the cells contacted with the first active agent at a first time point; measuring the concentration of the first active agent at a second time point; capturing a second optical signal from the cells contacted with the first active agent at a second time point; analyzing the first optical signal and the second optical signal to detect cell membrane motion of the cells; analyzing the cell membrane motion to quantify the viability of the cells following contact with the first active agent thereby detecting the drug induced damage at the second time point; measuring, calculating, and/or estimating the repair rate of the cells, therapeutic threshold, rate of sensitivity of therapy, and/or clonal composition of the tumor; repeating steps (a)-(i) with a second active agent; and calculating the synergistic effect of each active agent and/or pair or combination of active agents using an ex vivo mathematical malignancy advisor (EMMA) comprising a synergy augmented model (SAM). In some aspects following the repeat of steps (a)-(i) with a second active agent the method can comprise estimating sensitivity of each active agent using an ex vivo mathematical malignancy advisor (EMMA), followed by repeating steps (a)-(i) using the first and second active agents in combination to quantify the pharmacodynamic combination effect using synergy augmented model (SAM) rather than calculating the synergistic effect. In some aspects, the method can further comprise repeating steps (a)-(i) using the first and second active agents in combination.

6. Any cell type can be assayed by the disclosed methods. For example, the methods can be used to test for toxicity of a candidate agent on normal cells. Alternatively, the methods can be used to test cytotoxicity of a drug on abnormal cells, such as an antineoplastic drug on cancer cells. Therefore, in some examples, the cells are cancer cells, which can include solid tumor cells or hematological cancer cells (e.g., multiple myeloma).

7. The chamber of the disclosed method can be any chamber suitable to culture cells and allow imaging of the cells while in culture. For example, in some examples, the chamber is a microfluidic chamber. In some examples, the chamber is a well in a multi-well plate.

8. In some examples, the chamber can recapitulate the cell's natural microenvironment. This can involve the use of growth media, polymer substrates, feeder cells, stromal cells, growth factors, and the like. In some cases, the chamber recapitulates a cancer microenvironment. For example, culturing hematological cancer cells can involve a 3D reconstruction of the cancer microenvironment, e.g., including primary hematological cancer cells, extracellular matrix, patient-derived stroma, and growth factors.

9. In some examples, the active agent can comprise an anticancer agent, such as a chemotherapeutic agent. In some examples, the active agent can comprise a combination of active agents. For example, the anticancer agent can be a composition comprising melphalan, bortezomib, FAM-HYD-1, Marizomib (NPI-0052), Carfilzomib, Cytoxan, Dexamethasone, Thalidomide, Lenalidomide, Oprozomib, Panobinostat, Quisinostat, and Selinexor, or any combination thereof.

10. In some examples, the first optical signal, the second optical signal, or a combination thereof involves any optical microscopy illumination techniques suitable to detect cell membrane activity, such as a bright field illumination, dark field illumination, fluorescence microscopy, and phase contrast illumination.

11. In some examples, the cells of the method are obtained by collecting a sample from the subject and then isolating the cells from the sample. As an example, the sample can comprise a bone marrow aspirate where the cells are hematological cancer cells isolated from the aspirate, e.g., by flow cytometry using a cell surface cancer marker.

12. In some examples, the method can further comprise collecting and/or estimating parameters from the viability observations to generate a multi-parameter model that summarizes the response of a cancer in a subject to the active agent.

13. Also disclosed herein are methods for predicting a response of a subject to treatment with an active agent. The methods can comprise first preparing a three-dimensional dose-response curve by assessing the viability of cells from the subject in response to the active agent at a plurality of time points at a plurality of dosages. The method can then involve generating a multi-parameter model that summarizes the three-dimensional dose-response curve. The multi-parameter model can then be used to calculate the rate of accumulation of damage in the cells due to the active agent and the active agent-induced cell death due to the accumulated damage. In some embodiments, the number of distinct populations in the cells is a covariate in the multi-parameter model, so the method can involve determining the number of populations. The rate of accumulation of damage in the cells and the active agent-induced cell death due to the accumulated damage can then be extrapolated to predict a response of the subject to the active agent. For example, a three-dimensional dose-response curve based on 1, 2, 3, 4, 5, 6, 7, 8, 9, 10, 11, 12, 13, 14, 15, 16, 17, 18, 19, 20, 21, 22, 23, 24, 25, 28, 30, 32, 35, 36, 40, 42, 48 hours, 3, 4, 5, 6, 7, 8, 9, 10, 11, 12, 13, 14, 15, 16, 17, 18, 19, 20, 21, 22, 23, 24, 25, 26, 27, 28, 29, 30, 31, 35, 42, 49, 56, 60, 61, 62, or 90 days of viability data can be extrapolated to 1, 2, 3, 4, 5, 6, 7, 8, 9, 10, or more years of response by the subject. In some aspect, measurements can be obtained at least one time every 5, 10, 15, 20, 25, 30, 35, 40, 45, 50, 55, 60, 75, 90, 105, 120 minutes, 3, 4, 5, 6, 7, 8, 9, 10, 11, 12, 13, 14, 15, 16, 17 18, 19, 20, 21, 22, 23, or 24 hours.

14. In some examples, the methods disclosed herein can further comprise selecting a cancer treatment regimen for the subject based on predicted responses to 2, 3, 4, 5, 6, 7, 8, 9, 10, or more different active agents.

### III. BRIEF DESCRIPTION OF THE DRAWINGS

15. The accompanying drawings, which are incorporated in and constitute a part of this specification, illustrate several embodiments and together with the description illustrate the disclosed compositions and methods.

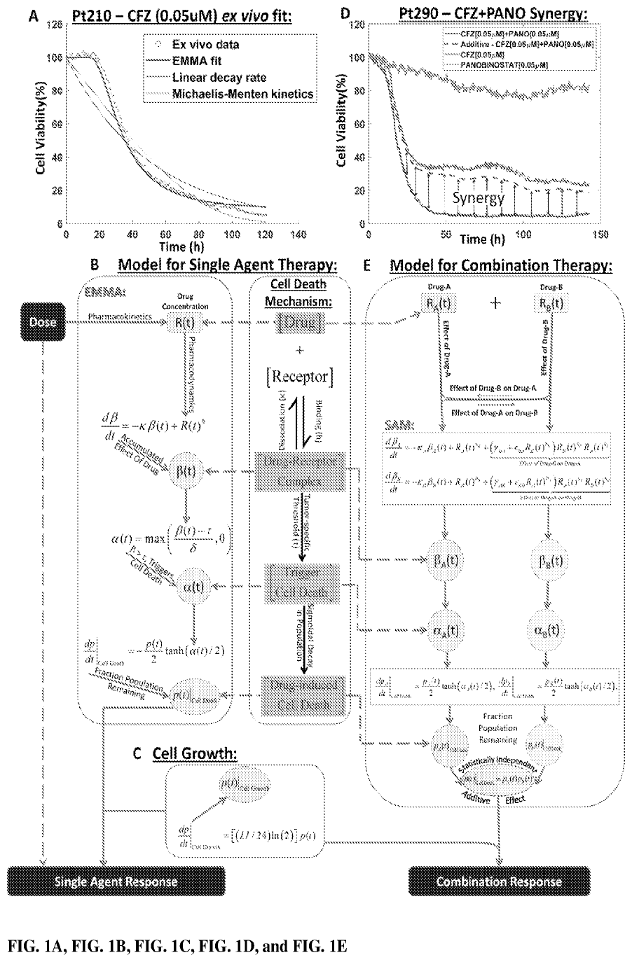

16. FIGS. 1A, 1B, 1C, 1D, and 1E show an overview of the modeling framework. FIG. 1A shows the response to therapy modeled as a second-order function of drug exposure: Pt210's ex vivo response to 0.05 μM of carfilzomib (blue scatter plot) was fit to a second-order sigmoidal function that accounts for tumor drug-specific threshold modeled as a precursor to cell death (EMMA, solid blue line). The EMMA model fit is compared to linear decay rate model (red solid line) and first-order Michaelis-Menten kinetic model (solid green line) to show that it is necessary to account for exposure-driven threshold that traditional models ignore. FIG. 1B shows an illustration of the drug-agnostic mechanism of response to single agent therapy: The drug-agnostic mechanism of cell death is based on drug occupancy theory, where the interaction of a drug with a receptor is governed by a reaction-kinetic equation that results in a drug-receptor complex (β), which initiates cell death beyond a clonal-specific threshold (τ) via cell death trigger (α). FIG. 1C shows a tumor growth model: A simple doubling time equation is used to estimate tumor growth, where 1% to 3% (LI) of the population is assumed to double every 24 hours. FIG. 1D shows that synergy is a dynamic phenomenon: Pt290's ex vivo response to 0.05 μM of carfilzomib (solid red line), 0.05 μM of panobinostat (solid green line), their combination (solid blue line), and the theoretical additive response (dashed blue line) computed from the two single agent response curves assuming Bliss independence are shown. The synergistic effect is measured as the difference in response between theoretical additive and the actual combination. It can be seen that synergistic interaction is a dynamic phenomenon and requires quantification using finely spaced temporal response data. FIG. 1E shows an illustration of the two-way pharmacodynamic modeling framework: The path from dose to response for a two-drug combination obeys the same mechanism of cell death as the single agent model but accounts for the two-way combination effect at the pharmacodynamic level by augmenting the reaction-kinetic equations used in computing the drug-receptor complex (β_{A} and (β_{B}) for single agents with a nonlinear combination effect term (β_{BA} and β_{AB}) as shown in the differential equations for β_{A} and β_{B}. The combination response is computed from the fraction population remaining estimates for the two drugs as if they were statistically independent. Abbreviations: CFZ, carfilzomib; EMMA, Ex Vivo Mathematical Malignancy Advisor; h/h_{A}/h_{B}, stoichiometric coefficient of the pharmacodynamic equation; LI, Labeling Index; M, Molar; Pt, Patient; p/p_{A}/p_{B}, predicted tumor burden; R/R_{A}/R_{B}, drug concentration; t, time; α/α_{A}/α_{B}, cell death trigger; β/β_{A}/β_{B}, drug-induced damage; δ, drug-specific factor; ε_{AB}/ε_{BA}, combination effect quadratic coefficient; γ_{AB}/γ_{BA}, combination effect linear coefficient; κ/κ_{A}/κ_{B}, cell dissociation coefficient in the pharmacodynamics equation; i, tumor-specific threshold.

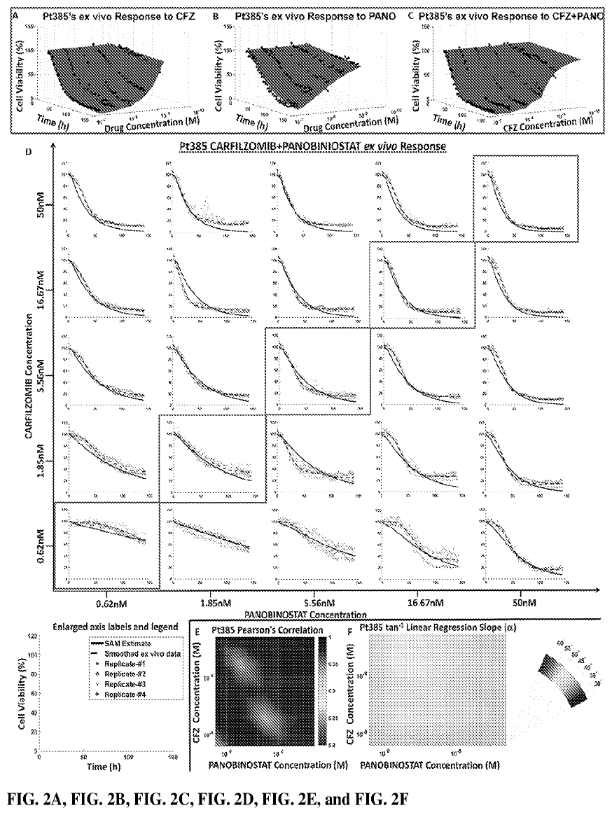

17. FIGS. 2A, 2B, 2C, 2D, 2E, and 2F show ex vivo validation of synergy augmented model (SAM). FIGS. 2A, 2B, and 2C show EMMA and SAM model parameters estimated from single agent and fixed concentration—ratio combination ex vivo response data: Pt385's ex vivo responses to carfilzomib (maximum concentration 0.05 μM) and panobinostat (maximum concentration 0.05 μM) as single agents is fit using EMMA as shown in 2A and 2B to estimate parameters that quantify the extent of response and tumor drug-specific heterogeneity. These parameters are used in conjunction with the combination response data (scatter plot) shown in 2C to estimate parameters that define the combination effect term in SAM. FIG. 2D shows checkered board assay response: A two-dimensional checkered board combination experiment is conducted to use the fixed concentration-ratio data to estimate SAM parameters and compare ex vivo model predictions with experimental results. Five three-fold serially diluted concentrations of each drug are combined yielding a 5×5 matrix of ex vivo combination response data with 4 replicates (shown as colored scatter plots) for each two-drug concentration duplet. The mean response of the four replicates is smoothed using LOWESS to estimate the smoothed ex vivo response data (black dashed line). The solid lines in the plots signify SAM model predictions. Enlarged axes labels and a legend are provided for each of the subplots in the checkered board assay. FIG. 2E shows SAM Validation—Pearson's correlation coefficients: Pearson's correlation coefficients (r) for each of the 25 two-drug concentration duplets are plotted on a log-log heat map, where the x and y axes show panobinostat and carfilzomib concentration, respectively, on log scales, and the color represents the r value. The model correlates very well with the data, with r values ranging from 0.93 to 1. FIG. 2F shows SAM Validation—Linear Regression: Similarly, a log-log heat map of the arc tangent of linear regression slope (α) for the 25 concentration duplets is shown to range from 45° to 50°, which implies that the model predictions agree very well with the ex vivo experimental combination response data. Abbreviations: CFZ, carfilzomib; h, hours; LOWESS, Locally Weighted Scatter Plot Smoothing; M, molar; PANO, panobinostat; Pt, patient; SAM, Synergy Augmented Model.

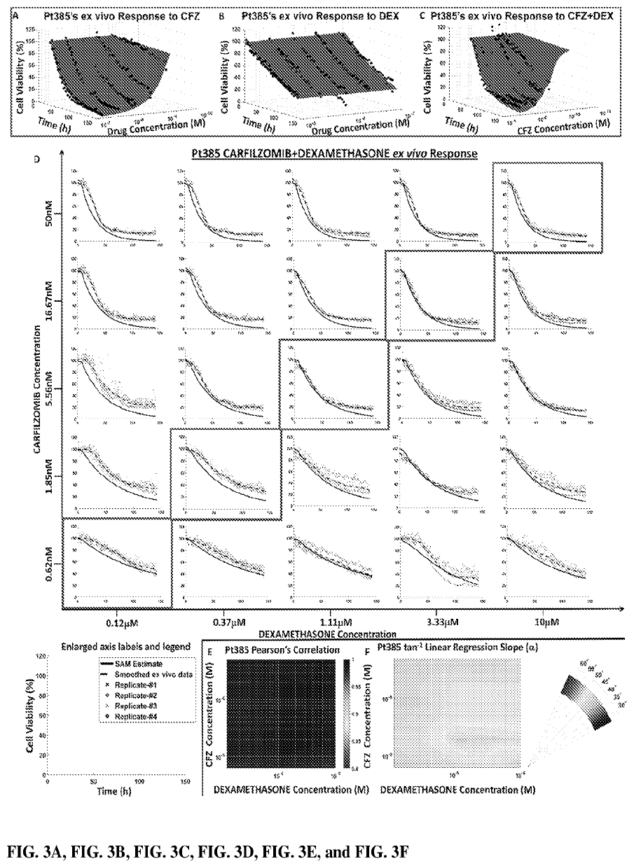

18. FIGS. 3A, 3B, 3C, 3D, 3E, and 3F show ex vivo validation of synergy augmented model (SAM). FIGS. 3A, 3B, and 3C show EMMA and SAM model parameters estimated from single agent and fixed concentration-ratio combination ex vivo response data: Pt385's ex vivo responses to carfilzomib (maximum concentration 0.05 μM) and dexamethasone (maximum concentration 10 μM) as single agents is fit using EMMA as shown in 3A and 3B to estimate parameters that quantify the extent of response and tumor-drug-specific heterogeneity. These parameters are used in conjunction with the combination response data (scatter plot) shown in 3C to estimate parameters that define the combination effect term in SAM. FIG. 3D shows checkered board assay response: A two-dimensional checkered board combination experiment is conducted to use the fixed concentration-ratio data to estimate SAM parameters and compare ex vivo model predictions with experimental results, where five three-fold serially diluted concentrations of each drug are combined yielding a 5×5 matrix of ex vivo combination response data with 4 replicates (shown as colored scatter plots) for each two-drug concentration duplet. The mean response of the four replicates is smoothed using Locally WEighted Scatter plot Smoothing (LOWESS) to estimate the smoothed ex vivo response data, which is indicated by a black dashed line. The solid lines in the plots signify SAM model predictions. Enlarged axes labels and a legend is provided for each of the sub-plots in the checkered board assay. FIG. 3E shows SAM Validation—Pearson's correlation coefficients: Pearson's correlation coefficients for each of the 25 two-drug concentration duplets are plotted on a log-log heat map, where the x and y axes show dexamethasone and carfilzomib concentration, respectively on log scales, and the color represents the value of the Pearson's correlation coefficient r. The model correlates very well with the data with r values ranging from 0.97 to 1. FIG. 3F shows SAM Validation—Linear Regression: Similarly, log-log heat map of the arc tangent of linear regression slope (α) for the 25 concentration duplets is shown to range from 45° to 50°, which implies that the model predictions agree very well with the ex vivo experimental combination response data. Abbreviations: CFZ, Carfilzomib; h, hours; M, Molar; DEX, Dexamethasone; Pt, Patient; SAM, Synergy Augmented Model.

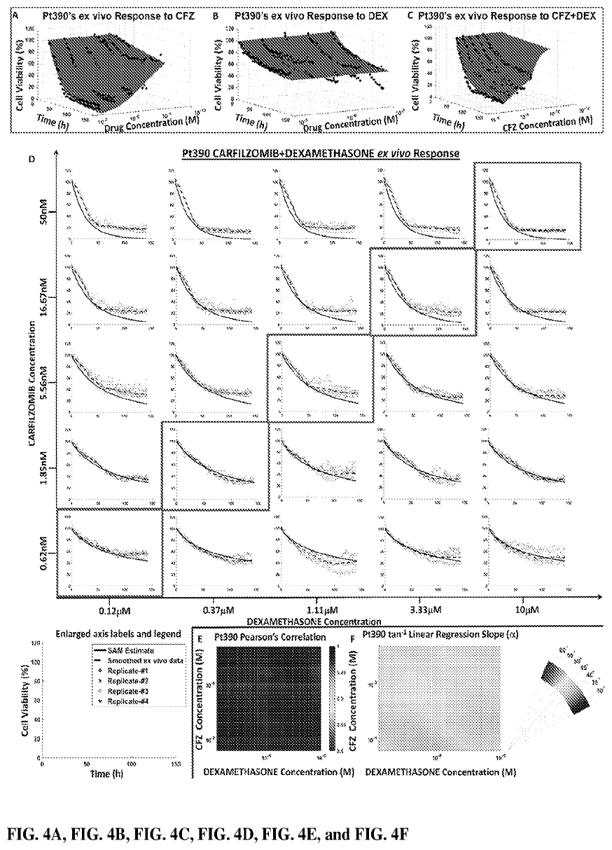

19. FIGS. 4A, 4B, 4C, 4D, 4E, and 4F show ex x vivo validation of synergy augmented model (SAM). FIGS. 4A, 4B, and 4C show EMMA and SAM model parameters estimated from single agent and fixed concentration-ratio combination ex vivo response data: Pt390's ex vivo responses to carfilzomib (maximum concentration 0.05 μM) and dexamethasone (maximum concentration 10 μM) as single agents is fit using EMMA as shown in 4A and 4B to estimate parameters that quantify the extent of response and tumor-drug-specific heterogeneity. These parameters are used in conjunction with the combination response data (scatter plot) shown in 4C to estimate parameters that define the combination effect term in SAM. FIG. 4D shows checkered board assay response: A two-dimensional checkered board combination experiment is conducted to use the fixed concentration-ratio data to estimate SAM parameters and compare ex vivo model predictions with experimental results, where five three-fold serially diluted concentrations of each drug are combined yielding a 5×5 matrix of ex vivo combination response data with 4 replicates (shown as colored scatter plots) for each two-drug concentration duplet. The mean response of the four replicates is smoothed using Locally WEighted Scatter plot Smoothing (LOWESS) to estimate the smoothed ex vivo response data, which is indicated by a black dashed line. The solid lines in the plots signify SAM model predictions. Enlarged axes labels and a legend is provided for each of the sub-plots in the checkered board assay. FIG. 4E shows SAM Validation—Pearson's correlation coefficients: Pearson's correlation coefficients for each of the 25 two-drug concentration duplets are plotted on a log-log heat map, where the x and y axes show dexamethasone and carfilzomib concentration, respectively on log scales, and the color represents the value of the Pearson's correlation coefficient r. The model correlates very well with the data with r values ranging from 0.97 to 1. FIG. 4F shows SAM

Validation—Linear Regression: Similarly, log-log heat map of the arc tangent of linear regression slope (α) for the 25 concentration duplets is shown to range from 42° to 50°, which implies that the model predictions agree very well with the ex vivo experimental combination response data Abbreviations: CFZ, Carfilzomib; h, hours; M, Molar; DEX, Dexamethasone; Pt, Patient; SAM, Synergy Augmented Model.

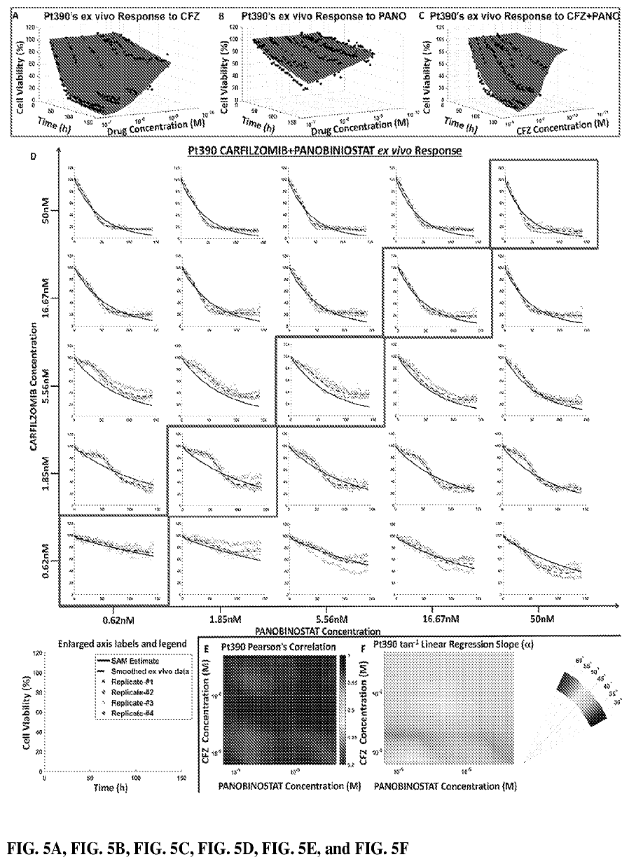

20. FIGS. 5A, 5B, 5C, 5D, 5E, and 5F show ex vivo validation of synergy augmented model (SAM). FIGS. 5A, 5B, and 5C show EMMA and SAM model parameters estimated from single agent and fixed concentration-ratio combination ex vivo response data: Pt385's ex vivo responses to carfilzomib (maximum concentration 0.05 μM) and panobinostat (maximum concentration 0.05 μM) as single agents is fit using EMMA as shown in 5A and 5B to estimate parameters that quantify the extent of response and tumor-drug-specific heterogeneity. These parameters are used in conjunction with the combination response data (scatter plot) shown in 5C to estimate parameters that define the combination effect term in SAM. FIG. 5D shows checkered board assay response: A two-dimensional checkered board combination experiment is conducted to use the fixed concentration-ratio data to estimate SAM parameters and compare ex vivo model predictions with experimental results, where five three-fold serially diluted concentrations of each drug are combined yielding a 5×5 matrix of ex vivo combination response data with 4 replicates (shown as colored scatter plots) for each two-drug concentration duplet. The mean response of the four replicates is smoothed using Locally WEighted Scatter plot Smoothing (LOWESS) to estimate the smoothed ex vivo response data, which is indicated by a black dashed line. The solid lines in the plots signify SAM model predictions. Enlarged axes labels and a legend is provided for each of the sub-plots in the checkered board assay. FIG. 5E shows SAM Validation—Pearson's correlation coefficients: Pearson's correlation coefficients for each of the 25 two-drug concentration duplets are plotted on a log-log heat map, where the x and y axes show panobinostat and carfilzomib concentration, respectively on log scales, and the color represents the value of the Pearson's correlation coefficient r. The model correlates very well with the data with r values ranging from 0.95 to 1. FIG. 5F shows SAM Validation—Linear Regression: Similarly, log-log heat map of the arc tangent of linear regression slope (α) for the 25 concentration duplets is shown to range from 40° to 50°, which implies that the model predictions agree very well with the ex vivo experimental combination response data. Abbreviations: CFZ, Carfilzomib; h, hours; M, Molar; PANO, Panobinostat; Pt, Patient; SAM, Synergy Augmented Model.

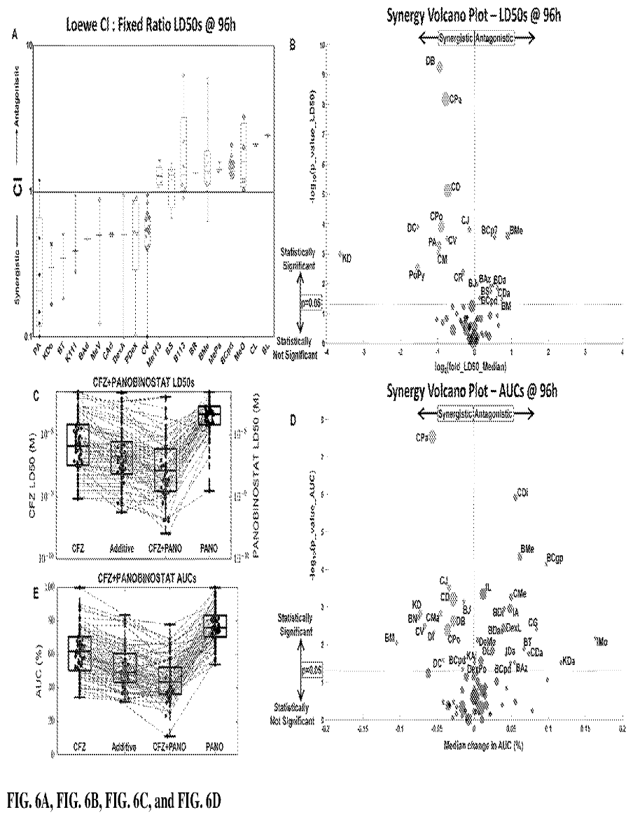

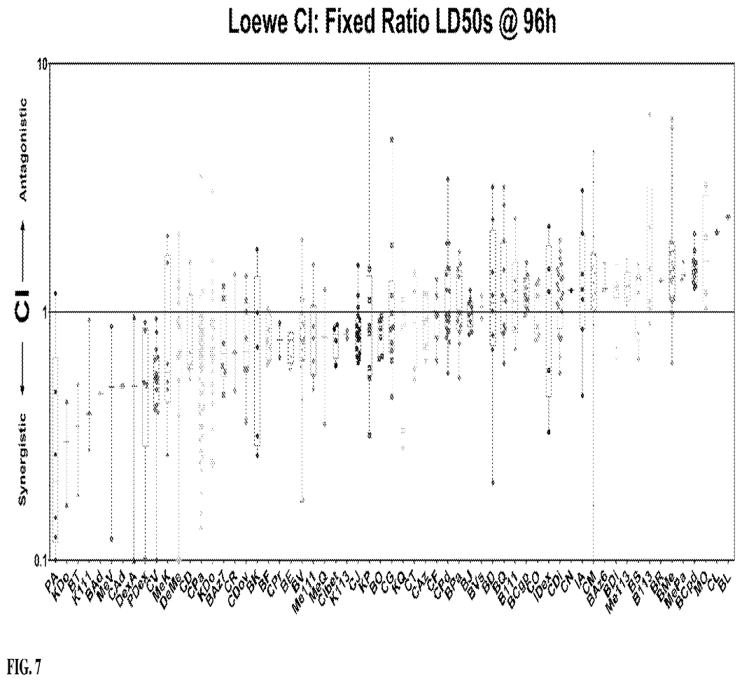

21. FIGS. 6A, 6B, 6C, and 6D show high-throughput combination screening based on ex vivo response measurements using CI, and a novel use of volcano plot to show statistical significance in synergy by LD50s and AUCs to demonstrate the relative merits and demerits of each method. FIG. 6A shows CIs presented as whisker box plots: CIs are shown as box-and-whisker plots for 20 combinations (the 10 most synergistic and antagonistic by median CI; the rest can be found in FIG. 7, which features 62 combinations) tested ex vivo, where the CI values are computed at LD50, 50% effect (cell kill), at 96 hours, estimated using EMMA and SAM models that capture tumor heterogeneity in a patient-specific manner. FIG. 6B shows high-throughput combination screening by LD50: High-throughput combination screenings for 56 combinations were tested using at least 10 patients' specimens each via a volcano plot. Each disc is a two-drug combination with an x-coordinate that represents the log₂ fold-change in LD50 at 96 hours for the median patient to signify the extent of combination effect, and the y-axis represents the −log₁₀ p-value for a paired t-test comparing the computed (from the two single-agent responses) additive responses (BLISS) to the combination responses to signify the statistical significance of the combination effect. Many combinations in 6A have sparse CI data, despite having ex vivo data from several patients (like BL and CL, which had 76 and 74 patients tested ex vivo), only one patient had a response, where both the single agents reached LD50. The volcano plot is a better approach to screen for synergistic combinations when using patient samples in diseases like MM as the combination response is compared to the additive response, which is computed from the response surfaces of the two single agents. This helps to consider combinations involving drugs that aren't equipotent in the high-throughput screen. FIG. 6C shows carfilzomib and panobinostat synergy by LD50 shown using a box-and-whisker plot: A box-and-whisker plot of LD50s for 60 MM patient samples treated ex vivo with carfilzomib (column 1), panobinostat (column 4), and their combination (column 3) is shown. The combination LD50s are compared to the additive LD50s (column 2) estimated from the additive response surface, which is the pointwise product of fraction population remaining at 96 hours for each of the two drugs. The red dashed lines indicate patients exhibiting synergy ex vivo for the combination, and the blue dashed lines indicate patients showing antagonism ex vivo. FIG. 6D shows carfilzomib and panobinostat synergy by AUC using a box-and-whisker plot: Similar to 6C, the additive response whisker box plot is compared to the combination response for the same 60 MM patients to estimate the P value for a paired t test. FIG. 6E shows high-throughput combination screening by AUC: Similar to 6B, a high-throughput combination screen is presented for 76 combinations, where the P value of the paired t test, estimated by comparing the additive and combination AUCs in 6D, is plotted along the y-coordinate and the x-coordinate shows the median change in AUC (%) between the additive and combination responses. The number of combinations and the criteria for studying them in 6A, 6B, and 6E. Abbreviations: B113, bortezomib and 113; BAd, bortezomib and adavosertib; BAz, bortezomib and AZ-628; BCgp, bortezomib and CGP-60474; BCp7, bortezomib and CP-724714; BCpd, bortezomib and CPD22; BDa, bortezomib and dabrafenib; BJ, bortezomib and JNK-IN-8; BL, bortezomib and lenalidomide; BM, bortezomib and MARK-INHIBITOR; BMe, bortezomib and melphalan; BN, bortezomib and NU-7441; BR, bortezomib and R406; BS, bortezomib and silmitasertib; BT, bortezomib and TAI-1; CAd, carfilzomib and adavosertib; CDa, carfilzomib and dabrafenib; CD, carfilzomib and dexamethasone; CDi, carfilzomib and dinaciclib; CG, carfilzomib and GDC-0980; CJ, carfilzomib and JNK-IN-8; CL, carfilzomib and lenalidomide; CM, carfilzomib and MARK-INHIBITOR; CMe, carfilzomib and melphalan; CPa, carfilzomib and panobinostat; CPo, carfilzomib and pomalidomide; CR, carfilzomib and R406; CV, carfilzomib and volasertib; DB, daratumumab and bortezomib; DC, daratumumab and carfilzomib; DI, daratumumab and ixazomib; DL, daratumumab and lenalidomide; DeMe, defactinib and melphalan; DexA, dexamethasone and ABT-199; DexL, dexamethasone and lenalidomide; DexPo, dexamethasone and pomalidomide; IA, ixazomib and ABT-199; IMo, ixazomib and motesanib; K111, selinexor and 111; KA1, selinexor and alisertib; KDa, selinexor and dabrafenib; KDo, selinexor and doxorubicin; Me113, melphalan and 113; MePa, melphalan and panobinostat; MeV, melphalan and VS4718; MeO, melphalan and ONX; PA, panobinostat and ABT-199; PDex, panobinostat and dexamethasone; PoPy, pomalidomide and pyrvinium; LD50, the dose that achieves 50% cell kill; AUC, average area under the dose-response curve over all time points; CI, Loewe's Combination Index; CFZ, carfilzomib; PANO, panobinostat; h, hours; M, molar.

22. FIG. 7 shows Loewe CI High-Throughput Combination Screen. FIG. 7 presents whisker box plots of CI values for 62 drug combinations arranged by their median CI from lowest to highest. A CI value below 1 indicates synergism and a value above 1 implies antagonism. The CI values are computed at 50% effect (cell kill), at 96 hours, estimated using EMMA and SAM models that capture tumor heterogeneity in a patient-specific manner. Combinations (n=Number of patients tested ex vivo): B111, Bortezomib and 111 (n=11); B113, Bortezomib and 113 (n=11); BAd, Bortezomib and Adavosertib (n=7); BAz6, Bortezomib and AZ-628 (n=10); BAz7, Bortezomib and AZD7762 (n=7); BCgp, Bortezomib and CGP-60474 (n=10); BCpd, Botezomib and CPD22 (n=11); BD, Bortezomib and Dexamethasone (n=65); BDi, Bortezomib and Dinaciclib (n=11); BE, Bortezomib and Elevenostat (n=7); BF, Bortezomib and F8 (n=7); BJ, Bortezomib and JNK-IN-8 (n=11); BK, Bortezomib and KPT-330 (n=6); BL, Bortezomib and Lenalidomide (n=65); BMe, Bortezomib and Melphalan (n=25); BO, Bortezomib and OTSSP167 (n=7); BPa, Bortezomib and Panobinostat (n=12); BQ, Bortezomib and Quisinostat (n=12); BR, Bortezomib and R406 (n=11); BS, Bortezomib and Silmitasertib (n=10); BT, Bortezomib and TAI-1 (n=11); BVs, Bortezomib and VS4718 (n=2); BV, Bortezomib and Volasertib (n=21)); CAd, Carfilzomib and Adavosertib (n=7); CAz, Carfilzomib and AZD7762 (n=7); CD, Carfilzomib and Dexamethasone (n=64); CDi, Carfilzomib and Dinaciclib (n=20); CDov, Carfilzomib and Dovitinib (n=10); CF, Carfilzomib and F8 (n=7); CG, Carfilzomib and GDC-0980 (n=17); CIbet, Carfilzomib and I-BET-762 (n=10); CJ, Carfilzomib and JNK-IN-8 (n=20); CL, Carfilzomib and Lenalidomide (n=64); CMe, Carfilzomib and Melphalan (n=24); CN, Carfilzomib and NU-7441 (n=20); CO, Carfilzomib and OTSSP167 (n=7); CPa, Carfilzomib and Panobinostat (n=63); CPd, Carfilzomib and CPD22 (n=20); CPr, Cobimetinib and Prexasertib (n=3); CR, Carfilzomib and R406 (n=20); CT, Carfilzomib and TAI-1 (n=20); CV, Carfilzomib and Volasertib (n=20 DexA, Dexamethasone and ABT-199 (n=22); DeMe, Defactinib and Melphalan (n=13); IA, Ixazomib and ABT-199 (n=22); ID, Ixazomib and Dexamethasone (n=42); K111, KPT-330 and 111 (n=6); K113, KPT-330 and 113 (n=6); KD, KPT-330 and Dexamethasone (n=24); KDo, KPT-330 and Doxorubicin (n=43); KP, KPT-330 and Panobinostat (n=10); KQ, KPT-330 and Quisinostat (n=10); Mel″, Melphalan and 111 (n=13); Me113, Melphalan and 113 (n=12); MB, MTI101 and Bortezomib (n=37); MeK, Melphalan and KPT-330 (n=8); MO, Melphalan and ONX (n=6); MePa, Melphalan and Panobinostat (n=4); MQ, Melphalan and Quisinostat (n=4); MV, Melphalan and VS4718 (n=2); PA, Panobinostat and ABT-199 (n=22); PDex, Panobinostat and Dexamethasone (n=42). Other Abbreviations: CI, Combination Index; LD50, Dose for 50% cell kill; h, hours.

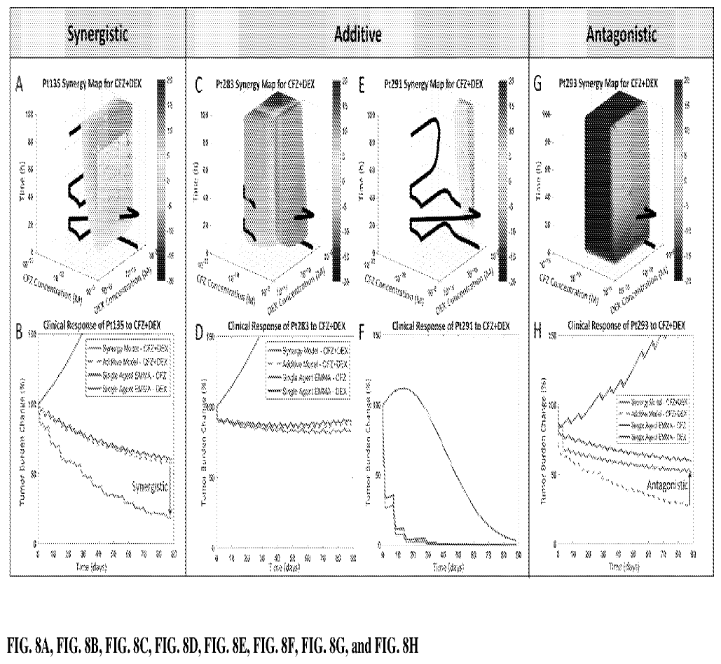

23. FIGS. 8A, 8B, 8C, 8D, 8E, 8F, 8G, and 8H show the interpatient heterogeneity in combination effect and clinical relevance of synergy. FIG. 8A shows a synergy map for Pt135's response to carfilzomib and dexamethasone: The theoretical additive response is estimated from the single agents' models (EMMA) and subtracted from the combination model (SAM) estimated ex vivo response for the first 96 hours over a wide range of concentrations/concentration ratios. The difference is presented as a heat map, where a benefit over additive (synergy) is indicated as ‘hot’ (yellow-red) and a loss in percent viability is marked as ‘cold’ (cyan-blue). The synergy map also features the pharmacokinetic curve of the standard of care therapeutic regimen for this combination. The relative residence period of the pharmacokinetic curve in the hot/cold regions qualitatively shows the extent of clinically relevant synergistic/antagonistic effect. FIG. 8B shows Pt135's predicted clinical response to carfilzomib and dexamethasone: The two single agent clinical response simulations (via EMMA), along with additive and combination response simulations (via SAM), are shown. The residence of the pharmacokinetic curve in the synergistic region is reflected in the clinical prediction. This analysis is repeated in 8C and 8D for Pt283, 8E and 8F for Pt291, and 8G and 8H for Pt293. These four patients were classified as early relapse/refractory at the time of their biopsy. In spite of their similar classification, the synergy maps and the clinical response simulations show significant variation. Abbreviations: CFZ, carfilzomib; DEX, dexamethasone; Pt, patient; h, hours; M, molar.

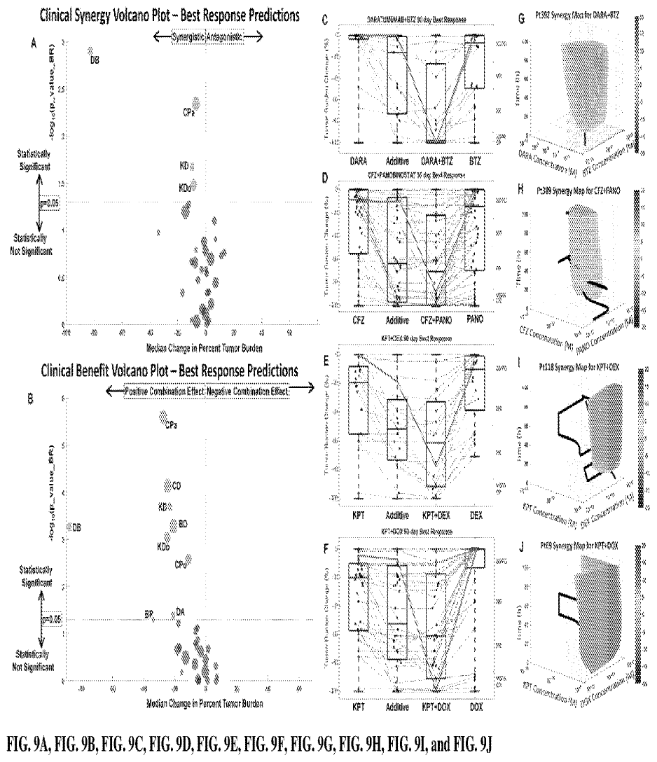

24. FIGS. 9A, 9B, 9C, 9D, 9E, 9F, 9G, 9H, 9I, and 9J show high-throughput combination screens of clinically synergistic and clinically beneficial combinations. FIG. 9A shows a clinical synergy via volcano plot: A volcano plot featuring 46 two-drug combination best response predictions computed from ex vivo experiments conducted across a cohort of 203 MM patients' specimens to screen for synergistic/antagonistic combinations that pass a paired t test between the combination clinical best response predictions and theoretical additive is shown. The theoretical additive response is the pointwise product of fraction cells surviving therapy (viability) for the two drugs as single agents. Further, best response is defined as the lowest percent population surviving therapy for 90 days. In contrast to LD50 and AUC, best response is a prediction of the clinical response from the model parameters (EMMA/SAM) estimated from ex vivo response data coupled with pharmacokinetic data from phase I clinical trials. The drugs that show clinically relevant synergy are shown as red discs. FIG. 9B shows the clinical benefit via volcano plot: Similarly, the combination clinical best response was compared to the more viable single agent to obtain the p-values and the median change in percent tumor burden. The more viable single agent response prediction is merely the best response of the drug that achieves greater percent cell kill. FIG. 9C shows daratumumab and bortezomib clinical synergy: The combination daratumumab and bortezomib are shown to be the most synergistic combination both by extent of synergism along the x-axis and by the likelihood of synergism on the y-axis. A whisker box plot is shown comparing the best response clinical predictions over a 90-day treatment period for the two single agents, the theoretical additive response prediction, and the combination. Red lines indicate synergism and blue lines indicate antagonism. The solid red line shows the patient with the most improvement over additive. FIG. 9D shows whisker box plots for carfilzomib and panobinostat; FIG. 9E shows whisker box plot for selinexor and dexamethasone; FIG. 9F shows whisker box plot for selinexor and liposomal doxorubicin. The solid red line in each of 9C-9F is the patient with the most clinically-relevant predicted synergistic effect. 9G-9J, Ex vivo synergy maps: Heat maps are used to show regions of ex vivo synergy/antagonism. Regions of red indicate synergy, blue denote antagonism, and empty spaces represent additivity for the four statistically significant combinations shown in A. The criteria for studying the 46 combinations featured in A and B is presented herein. Abbreviations: BD, Bortezomib and Dexamethasone; BP, bortezomib and pomalidomide; CD, carfilzomib and dexamethasone; CPa, carfilzomib and panobinostat; CPo, carfilzomib and pomalidomide; DA, dexamethasone and ABT-199; DB, daratumumab and bortezomib; KD, KPT-330 and dexamethasone; KDo, KPT-330 and doxorubicin; BR, best response; DARA, daratumumab; BTZ, bortezomib; CFZ, carfilzomib; PANO, panobinostat; KPT, selinexor; DEX, dexamethasone; DOX, doxorubicin; h, hours; M, molar.

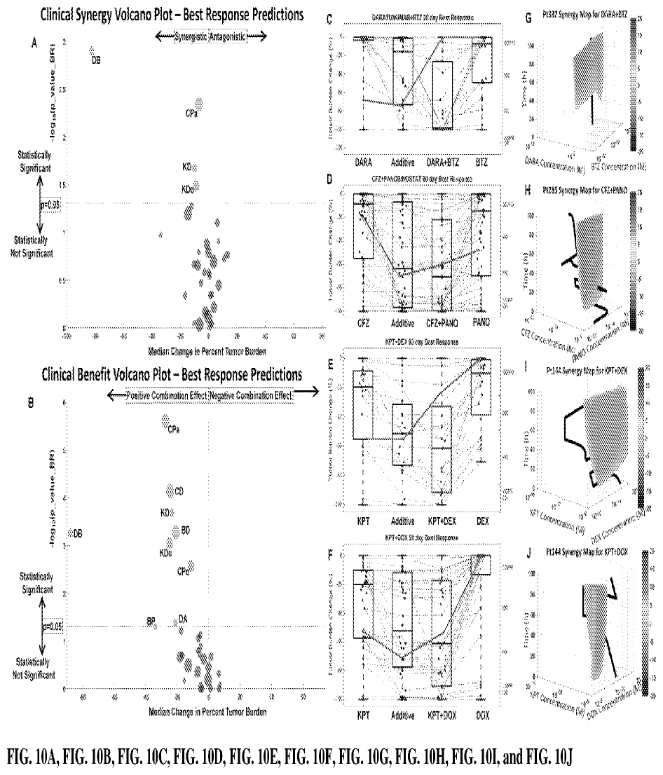

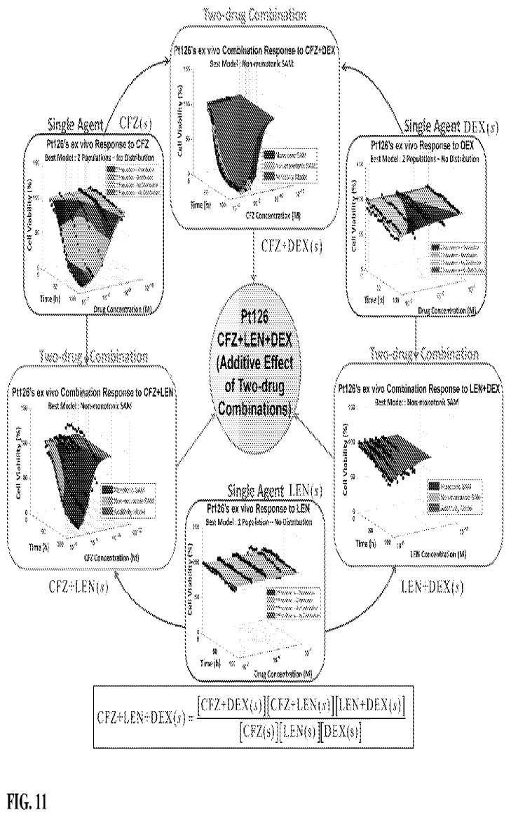

25. FIGS. 10A, 10B, 10C, 10D, 10E, 10F, 10G, 10H, 10I, and 10J show high-throughput combination screens of clinically synergistic and clinically beneficial combinations. FIG. 10A shows clinical Synergy via Volcano plot: A volcano plot featuring 46 two-drug combination best response predictions computed from ex vivo experiments conducted across a cohort of 203 MM patients' specimen to screen for synergistic/antagonistic combinations that pass a paired t-test between the combination clinical best response predictions and theoretical additive, where the theoretical additive best response is the pointwise product of fraction cells surviving therapy (viability) for the two drugs to compute viability for the theoretical additive response. Further, best response is defined as the lowest percent population surviving therapy for 90 days. In contrast to LD50 and AUC, best response is a prediction of the clinical response from the model parameters (EMMA/SAM) estimated from ex vivo response data coupled with pharmacokinetic data from phase I clinical trials. The drugs that show clinically-relevant synergy are shown as red discs. FIG. 10B shows the clinical Benefit via Volcano plot: Similarly, the combination clinical best response was compared to the more viable single agent to obtain the p-values and the median change in percent tumor burden. The more viable single agent response prediction is merely the best response of the drug that achieves greater percent cell kill. FIG. 10C shows Daratumumab and Bortezomib Clinical Synergy: The combination Daratumumab and Bortezomib are shown to be the most synergistic combination both by extent of synergism along the x-axis and the likelihood of synergism on the y-axis. A whisker box plot is shown comparing the best response clinical predictions over a 90 day treatment period for the two single agents, the theoretical additive response prediction, and the combination. All the red lines indicate synergism and the blue line implies antagonism. The solid blue line shows the patient with the most antagonism. Similarly, 10D presents whisker box plots for Carfilzomib and Panobinostat, 10E presents whisker box plot for Selinexor and Dexamethasone, and 10F presents whisker box plot for Selinexor and liposomal Doxorubicin. The solid blue line in each of these four figures is the patient with the most clinically-relevant predicted synergistic effect. 10G-10J present the ex vivo synergy maps: Heat maps are used to show regions of ex vivo synergy/antagonism, where regions of red indicate synergy, blue denote antagonism, and empty spaces represent additivity for the four statistically significant combinations shown in A. Combinations: BD, Bortezomib and Dexamethasone; BP, Bortezomib and Pomalidomide; CD, Carfilzomib and Dexamethasone; CPa, Carfilzomib and Panobinostat; CPo, Carfilzomib and Pomalidomide; DA, Dexamethasone and ABT-199; DB, Daratumumab and Bortezomib; KD, KPT-330 and Dexamethasone; KDo, KPT-330 and Doxorubicin. Other Abbreviations: BR, Best Response; DARA, Daratumumab; BTZ, Bortezomib; CFZ, Carfilzomib; PANO, Panobinostat; KPT, Selinexor; DEX, Dexamethasone; DOX, Doxorubicin; h, hours; M, Molar.

26. FIG. 11 shows computing Three-drug Combination Response from Two-drug Combination Responses: A graphic showing how a three-drug combination response is computed using three single agents' (in red boxes), and three two-drug combinations' (in green boxes) ex vivo response surfaces is shown. Single agent ex vivo response data is used to estimate parameters for the single agent EMMA model that measures chemosensitivity of a drug by accounting for intratumoral heterogeneity. The two-drug combination ex vivo response data is used in conjunction with the EMMA model parameters from the two single agents to estimate parameters that govern the two-drug combination effect. This is shown by arrows connecting the two constituent single agents' ex vivo response surfaces (in red boxes) and their combination ex vivo response surface (green box). The three two-drug combination responses are then used to compute the three-drug combination response for Pt126's response to CFZ+LEN+DEX assuming that the second-order combination effect interactions are additive. Abbreviations: CFZ, Carfilzomib; CFZ+DEX, Carfilzomib/Dexamethasone; CFZ+LEN, Carfilzomib/Lenalidomide; CFZ+LEN+DEX, Carfilzomib/Lenalidomide/Dexamethasone; DEX, Dexamethasone; h, hours; LEN, Lenalidomide; M, Molar; Pt, Patient; s, Laplace variable; SAM, Synergy Augmented Model.

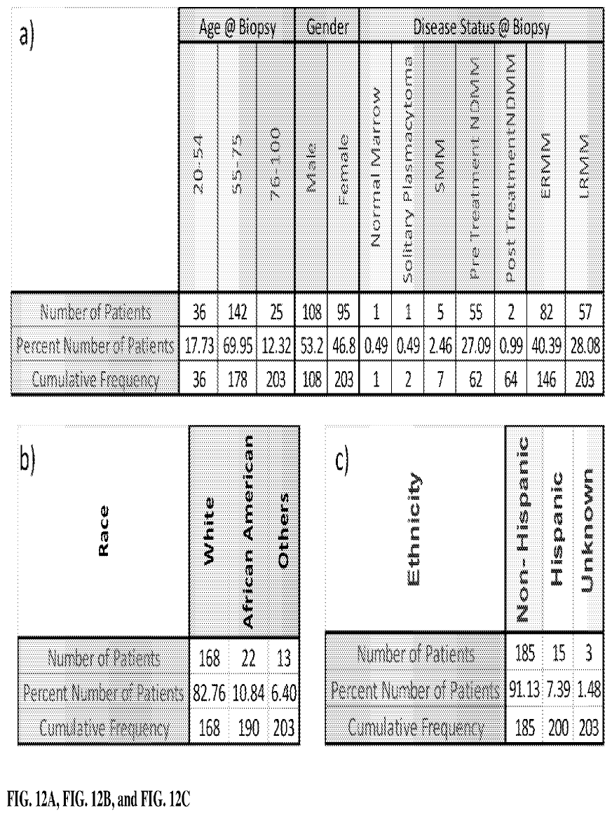

27. FIGS. 12A, 12B, and 12C show demographic information for the 203 patients tested using the ex vivo modeling framework: FIG. 12A shows demographics by age, gender, and disease status: A summary of patient demographics for all the patients tested ex vivo by age, gender, and disease status is presented. Age at biopsy is classified into three bins; 20-54 years, 55-75 years, and 76-100 years. Gender identification is Male/Female. Disease status at biopsy is classified into normal marrow, solitary plasmacytoma, smoldering multiple myeloma (SMM), pre-treatment newly diagnosed multiple myeloma (NDMM), post-treatment NDMM, early relapsed/refractory multiple myeloma (ERMM), and late relapsed/refractory multiple myeloma (LRMM). FIG. 12B shows demographics by race: Patients are classified by race into three categories; white, African American, and others. FIG. 12C shows demographics by ethnicity: Patients are classified by ethnicity into three categories; non-Hispanic, Hispanic, and unknown.

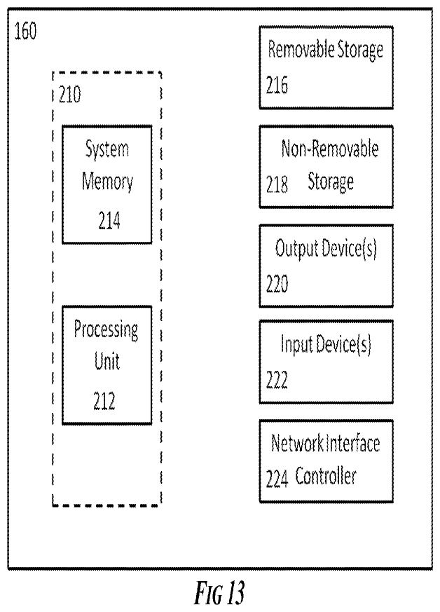

28. FIG. 13 shows a schematic of an exemplary computing device.

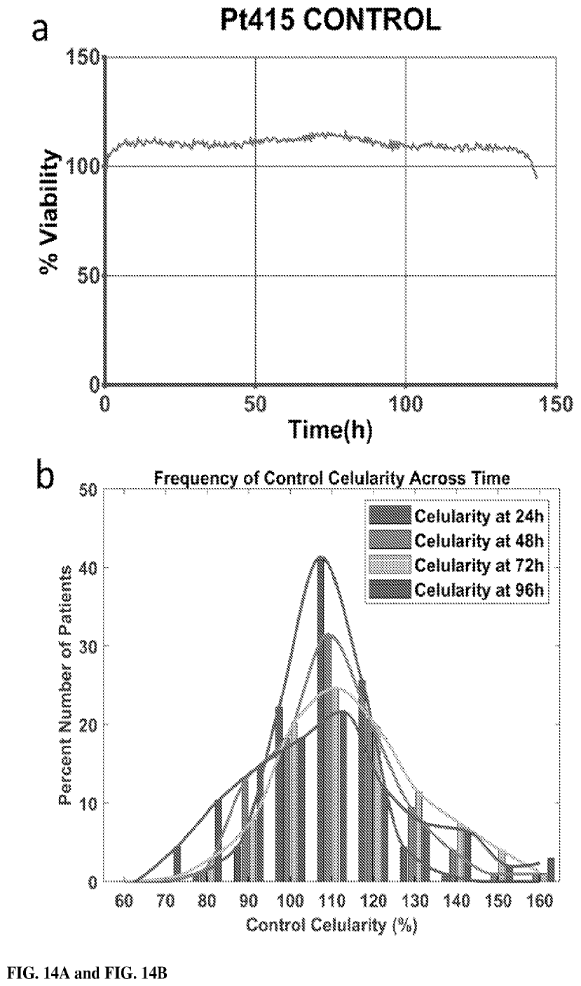

29. FIGS. 14A and 14B show the percent viability of primary MM cells in an ex vivo reconstruction of the bone marrow for upto 6 days. FIG. 14A shows the percent viability of Pt415, a 65 year old female early relapsed/refractory MM patient, for 6 days. Pt415's bone marrow specimen was enriched for CD138+ cells, which are co-cultured in an ex vivo reconstruction of the bone marrow for upto 6 days as described in Materials and Methods. Live imaging of the multi-well plate resulted in percent viability measurements, once every 30 mins. These percent viability measures are shown in a. FIG. 14B shows a grouped bar plot shows a histogram of primary MM control cellularity at 24, 48, 72, and 96 hours indicates stability of MM cell population ex vivo. A grouped bar plot presents a histogram of cellularity every 24 hours during the ex vivo assay for all the 203 MM patient specimens. A majority of patient specimens stay within the 100 to 120 percent of initial cellularity, some decay upto 70% and the other grow steadily to 160%. However, the effect of these differences is mitigated by normalizing the response measurements with the control. These normalized response measurements are used to inform model parameter estimation, and eventually clinical predictions.

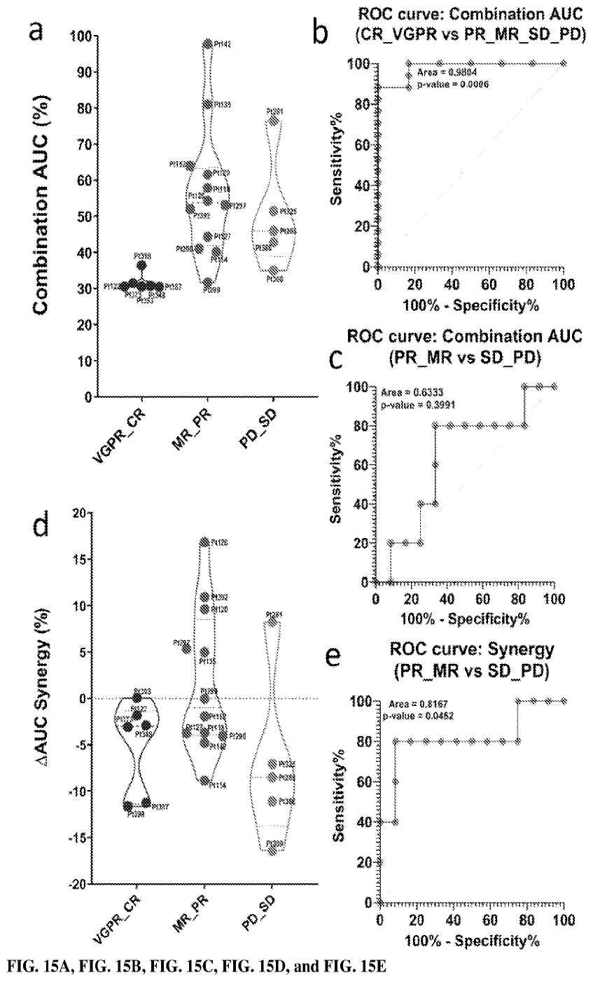

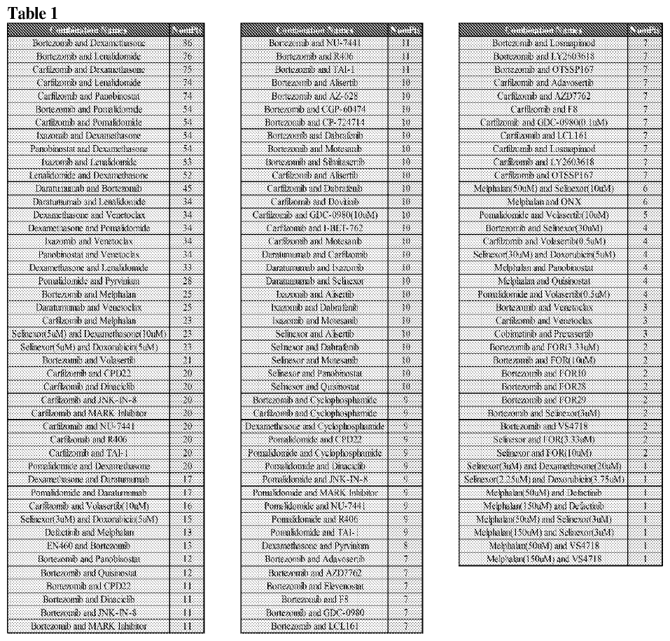

30. FIGS. 15A, 15B, 15C, 15D, and 15E show ex vivo Synergy—A Classifier of Clinical Response. FIG. 15A shows a violin plot of Combination AUC: The ex vivo AUC values for the combination received by 23 patients immediately following their biopsies are computed using the approach described in FIG. 15, and presented as a violin plot, where the patients are categorized into three groups: those who responded with a very good partial response (VGPR) or complete response (CR), those whose response was either minimal, or partial (MR/PR), and those patients who had either stable (SD) or progressive disease (PD) when treated, following the biopsy, with the same combination therapy tested ex vivo. FIG. 15B shows a Receiver Operating Characteristic (ROC) Curve for combination AUC in classifying between CR/VGPR and PR/MR/SD/PD patients: The ROC curve shows that the “combination AUC” is an excellent classifier between CR/VGPR, and PR or worse response stratifications. The area under ROC is nearly 1 (0.9804) and has a p-value of 0.0006 (for a t-test with the null hypothesis that area is 0.5). FIG. 15C shows a ROC Curve for combination AUC in classifying between PR/MR and SD/PD patients: However, this metric alone is a poor classifier to distinguish between patients with MR/PR and PD/SD. The area under ROC is 0.6333 and has a p-value of 0.3991 (for a t-test with the null hypothesis that area is 0.5). FIG. 15D shows a violin plot of difference in AUC between additive and combination responses: The difference in ex vivo combination and additive AUC values (signifying the benefit due to synergistic interactions when positive and the effect of antagonism when negative) are presented for the same 23 patients shown in a and are categorized into the same three groups. VGPR_CR and PD_SD columns are predominantly antagonistic as these two groups of patients either see an excellent response due to the efficacy of one of the single agents that there is no scope for improvement, or experience progression due to likely concurrent mechanisms of resistance that could potentially lead to blocking pathways for synergistic interaction. The MR_PR column however seems to show most variability in terms of combination effect. Using ΔAUC Synergy as a classifier between MR_PR and PD_SD stratifications could help address the issue in 15C. FIG. 15E shows a ROC Curve for A AUC Synergy in classifying between PR/MR and SD/PD patients: The ROC curve shows that A AUC Synergy is a good classifier between PR/MR, and SD/PD stratifications. The area under ROC is 0.8167 and has a statistically significant p-value of 0.0452 (for a t-test with the null hypothesis that area is 0.5).

### IV. DETAILED DESCRIPTION

31. Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

32. As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

33. Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

34. “Optional” or “optionally” means that the subsequently described event or circumstance may or may not occur, and that the description includes instances where said event or circumstance occurs and instances where it does not.

35. A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

36. “Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

37. By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

38. “Treat,” “treating,” “treatment,” and grammatical variations thereof as used herein, include the administration of a composition with the intent or purpose of partially or completely preventing, delaying, curing, healing, alleviating, relieving, altering, remedying, ameliorating, improving, stabilizing, mitigating, and/or reducing the intensity or frequency of one or more a diseases or conditions, a symptom of a disease or condition, or an underlying cause of a disease or condition. Treatments according to the invention may be applied preventively, prophylactically, palliatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for day(s) to years prior to the manifestation of symptoms of an infection.

39. By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

40. “Biocompatible” generally refers to a material and any metabolites or degradation products thereof that are generally non-toxic to the recipient and do not cause significant adverse effects to the subject.

41. “Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

42. A “control” is an alternative subject or sample used in an experiment for comparison purposes. A control can be “positive” or “negative.”

43. The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

44. “Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

45. A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

46. “Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

47. “Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

48. “Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

49. “Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

50. The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

51. Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Methods of Using the Compositions

52. There are multiple methodologies that exist in the art to identify combination therapies and establish a suitable treatment regimen for a subject. Often times, such methodologies look at the cell killing of the drug combination therapy relative to either or each of the drugs in the combination alone. A combination that shows more killing than the additive amount of killing of the drugs individually is seen as being a synergistic combination. The problem with such a methodology is that it does not take into account biological dynamics in the subject or pharmacological dynamics of the drugs used in the combination.

53. Disclosed herein are systems and methods comprising a computational model of clinical response. In some examples, the systems and methods can combine the dose-response platform, for ex vivo screening of drugs and the computational model of clinical response. In some examples, the ex vivo component can include a 3D reconstruction of a cancer microenvironment, e.g., including primary cancer cells, extracellular matrix, and patient-derived stroma and growth factors. In some examples, live microscopy and digital image analysis can be used to detect cell death events in different drug concentrations, which can then be used to generate dose-response surfaces. In some examples, an evolutionary computational model designed to simulate how a heterogeneous population of cancer cells responds to therapy is used as an in silico component of the methods described herein. From the ex vivo data, the model can identify the size and chemosensitivity of subpopulations within the patient's tumor burden, measure the concentration of a drug over time, the drug induced damage over time, the repair rate, and the effect that each drug in a combination therapy has on each other, and simulate how the tumor would respond to the drug(s) in physiological conditions in a clinical regimen.

54. Pre-clinical assays specifically designed to generate data to parameterize such computational models, preferably comply with one or more of the following conditions: (a) compatibility with patient primary cancer cells; (b) recapitulate the tumor microenvironment, namely extra-cellular matrix and stroma; (c) be non-destructive, so longitudinal studies can be performed, incorporating the temporal dimension; (d) use as few cells per experimental condition as possible, so each patient sample could be tested against a panel of chemotherapeutic agents, in different environmental conditions; and (e) the data generated should result in testable clinical predictions, such as the depth of response and/or progression-free survival (PFS).

55. Disclosed herein are non-destructive methods for quantifying cell viability. In some examples, the method can comprise culturing a plurality of cells from a subject in a chamber; capturing a first optical signal from the cells at a first time point; capturing a second optical signal from the cells at a second time point; analyzing the first optical signal and the second optical signal to detect cell membrane motion of the cells; and analyzing the cell membrane motion to quantify the viability of the cells. In some embodiments, the method is used to quantify cell viability after the cells have been exposed to an active agent. Therefore, in some embodiments, the method further comprises contacting the cells with an active agent and then quantifying the effect of the active agent on cell membrane motion (i.e., viability).

56. In some examples, the cells can comprise cancer. Examples include cancer and/or tumors of the anus, bile duct, bladder, bone, bone marrow, bowel (including colon and rectum), breast, eye, gall bladder, kidney, mouth, larynx, esophagus, stomach, testis, cervix, head, neck, ovary, lung, mesothelioma, neuroendocrine, penis, skin, spinal cord, thyroid, vagina, vulva, uterus, liver, muscle, pancreas, prostate, blood cells (including lymphocytes and other immune system cells), and brain. Other examples of cancers include adrenocortical carcinoma, adrenocortical carcinoma, cerebellar astrocytoma, basal cell carcinoma, bile duct cancer, bladder cancer, bone cancer, brain tumor, breast cancer, Burkitt's lymphoma, carcinoid tumor, central nervous system lymphoma, cervical cancer, chronic myeloproliferative disorders, colon cancer, cutaneous T-cell lymphoma, endometrial cancer, ependymoma, esophageal cancer, gallbladder cancer, gastric (stomach) cancer, gastrointestinal carcinoid tumor, germ cell tumor, glioma, hairy cell leukemia, head and neck cancer, hepatocellular (liver) cancer, hypopharyngeal cancer, hypothalamic and visual pathway glioma, intraocular melanoma, retinoblastoma, islet cell carcinoma (endocrine pancreas), laryngeal cancer, lip and oral cavity cancer, liver cancer, medulloblastoma, Merkel cell carcinoma, squamous neck cancer with occult mycosis fungoides, myelodysplastic syndromes, myelogenous leukemia, nasal cavity and paranasal sinus cancer, nasopharyngeal cancer, neuroblastoma, non-small cell lung cancer, oral cancer, oropharyngeal cancer, osteosarcoma, ovarian cancer, pancreatic cancer, paranasal sinus and nasal cavity cancer, parathyroid cancer, penile cancer, pheochromocytoma, pineoblastoma and supratentorial primitive neuroectodermal tumor, pituitary tumor, plasma cell neoplasm/multiple myeloma, pleuropulmonary blastoma, prostate cancer, rectal cancer, renal cell (kidney) cancer, retinoblastoma, rhabdomyosarcoma, salivary gland cancer, Ewing's sarcoma, soft tissue sarcoma, Sezary syndrome, skin cancer, small cell lung cancer, small intestine cancer, supratentorial primitive neuroectodermal tumors, testicular cancer, thymic carcinoma, thymoma, thyroid cancer, transitional cell cancer of the renal pelvis and ureter, trophoblastic tumor, urethral cancer, uterine cancer, vaginal cancer, vulvar cancer, Waldenström's macroglobulinemia, and Wilms' tumor.

57. In some examples, the cancer can comprise a hematological cancer. Hematological cancers are the types of cancer that affect blood, bone marrow and lymph nodes. As the three are intimately connected through the immune system, a disease affecting one of the three will often affect the others as well. Hematological cancers may derive from either of the two major blood cell lineages: myeloid and lymphoid cell lines. The myeloid cell line normally produces granulocytes, erythrocytes, thrombocytes, macrophages and mast cells; the lymphoid cell line produces B, T, NK and plasma cells. Lymphomas, lymphocytic leukemias, and myeloma are from the lymphoid cell line, while acute and chronic myelogenous leukemia, myelodysplastic syndromes and myeloproliferative diseases are myeloid in origin.

58. In some examples, the cancer can comprise multiple myeloma. Multiple myeloma is the second most common hematological cancer in the United States, and constitutes 1% of all cancers. Specifically, multiple myeloma is a cancer of plasma cells, a type of white blood cell normally responsible for producing antibodies. In multiple myeloma, collections of abnormal plasma cells accumulate in the bone marrow, where they interfere with the production of normal blood cells. Kidney problems, bone lesions and hypercalcemia are common complications associated with multiple myeloma. Myeloma develops in 1-4 per 100,000 people per year. It is more common in men, and is twice as common in African-Americans as it is in European-Americans. With conventional treatment, median survival is 3-4 years, which may be extended to 5-7 years or longer with advanced treatments.

59. The chamber can comprise any chamber consistent with the methods described herein. Examples of suitable chambers can include, but are not limited to, petri dishes, laboratory flasks (e.g., Erlenmeyer flasks, beakers, conical flasks, round bottom flasks, culture flasks), microfluidic chambers, multi-well-pates, and the like. In some examples, the chamber can comprise any chamber that allows for bright field imaging. In some examples, the chamber can comprise a microfluidic chamber. In some examples, the chamber can comprise a well in a multi-well plate.

60. In some examples, the chamber can recapitulate the cancer microenvironment. In some examples, the culturing a plurality of cancer cells from a subject in a chamber can include a 3D reconstruction of the cancer microenvironment, e.g., including primary cancer cells, extracellular matrix, and patient-derived stroma and growth factors.

61. The active agent can comprise a wide variety of drugs, including antagonists, for example enzyme inhibitors, and agonists, for example a transcription factor which results in an increase in the expression of a desirable gene product (although as will be appreciated by those in the art, antagonistic transcription factors can also be used), are all included. In addition, the active agent includes those agents capable of direct toxicity and/or capable of inducing toxicity towards healthy and/or unhealthy cells in the body. Also, the active agent can be capable of inducing and/or priming the immune system against potential pathogens.

62. The active agent can, for example, comprise an anticancer agent, antiviral agent, antimicrobial agent, anti-inflammatory agent, immunosuppressive agent, anesthetics, or any combination thereof.

63. In some examples, the active agent can comprise an anticancer agent. Examples of anticancer agents include 13-cis-Retinoic Acid, 2-Amino-6-Mercaptopurine, 2-CdA, 2-Chlorodeoxyadenosine, 5-fluorouracil, 6-Thioguanine, 6-Mercaptopurine, Accutane, Actinomycin-D, Adriamycin, Adrucil, Agrylin, Ala-Cort, Aldesleukin, Alemtuzumab, Alitretinoin, Alkaban-AQ, Alkeran, All-transretinoic acid, Alpha interferon, Altretamine, Amethopterin, Amifostine, Aminoglutethimide, Anagrelide, Anandron, Anastrozole, Arabinosylcytosine, Aranesp, Aredia, Arimidex, Aromasin, Arsenic trioxide, Asparaginase, ATRA, Avastin, BCG, BCNU, Bevacizumab, Bexarotene, Bicalutamide, BiCNU, Blenoxane, Bleomycin, Bortezomib, Busulfan, Busulfex, C225, Calcium Leucovorin, Campath, Camptosar, Camptothecin-11, Capecitabine, Carac, Carboplatin, Carfilzomib, Carmustine, Carmustine wafer, Casodex, CCNU, CDDP, CeeNU, Cerubidine, cetuximab, Chlorambucil, Cisplatin, Citrovorum Factor, Cladribine, Cortisone, Cosmegen, CPT-11, Cyclophosphamide, Cytadren, Cytarabine, Cytarabine liposomal, Cytosar-U, Cytoxan, Dacarbazine, Dactinomycin, Daratumumab, Darbepoetin alfa, Daunomycin, Daunorubicin, Daunorubicin hydrochloride, Daunorubicin liposomal, DaunoXome, Decadron, Delta-Cortef, Deltasone, Denileukin diftitox, DepoCyt, Dexamethasone, Dexamethasone acetate, Dexamethasone sodium phosphate, Dexasone, Dexrazoxane, DHAD, DIC, Diodex, Docetaxel, Doxil, Doxorubicin, Doxorubicin liposomal, Droxia, DTIC, DTIC-Dome, Duralone, Efudex, Eligard, Ellence, Eloxatin, Elspar, Emcyt, Epirubicin, Epoetin alfa, Erbitux, Erwinia L-asparaginase, Estramustine, Ethyol, Etopophos, Etoposide, Etoposide phosphate, Eulexin, Evista, Exemestane, Fareston, Faslodex, Femara, Filgrastim, Floxuridine, Fludara, Fludarabine, Fluoroplex, Fluorouracil, Fluorouracil (cream), Fluoxymesterone, Flutamide, Folinic Acid, FUDR, Fulvestrant, G-CSF, Gefitinib, Gemcitabine, Gemtuzumab ozogamicin, Gemzar, Gleevec, Lenalidomide, Lupron, Lupron Depot, Matulane, Maxidex, Mechlorethamine, -Mechlorethamine Hydrochlorine, Medralone, Medrol, Megace, Megestrol, Megestrol Acetate, Melphalan, Mercaptopurine, Mesna, Mesnex, Methotrexate, Methotrexate Sodium, Methylprednisolone, Mylocel, Letrozole, Neosar, Neulasta, Neumega, Neupogen, Nilandron, Nilutamide, Nitrogen Mustard, Novaldex, Novantrone, Octreotide, Octreotide acetate, Oncospar, Oncovin, Ontak, Onxal, Oprevelkin, Oprozomib, Orapred, Orasone, Oxaliplatin, Paclitaxel, Pamidronate, Panobinostat, Panretin, Paraplatin, Pediapred, PEG Interferon, Pegaspargase, Pegfilgrastim, PEG-INTRON, PEG-L-asparaginase, Phenylalanine Mustard, Platinol, Platinol-AQ, Pomalidomide, Prednisolone, Prednisone, Prelone, Procarbazine, PROCRIT, Proleukin, Prolifeprospan 20 with Carmustine implant, Purinethol, Quisinostat, Raloxifene, Rheumatrex, Rituxan, Rituximab, Roveron-A (interferon alfa-2a), Rubex, Rubidomycin hydrochloride, Sandostatin, Sandostatin LAR, Sargramostim, Selinexor, Solu-Cortef, Solu-Medrol, STI-571, Streptozocin, Tamoxifen, Targretin, Taxol, Taxotere, Temodar, Temozolomide, Teniposide, TESPA, Thalidomide, Thalomid, TheraCys, Thioguanine, Thioguanine Tabloid, Thiophosphoamide, Thioplex, Thiotepa, TICE, Toposar, Topotecan, Toremifene, Trastuzumab, Tretinoin, Trexall, Trisenox, TSPA, VCR, Velban, Velcade, VePesid, Vesanoid, Viadur, Vinblastine, Vinblastine Sulfate, Vincasar Pfs, Vincristine, Vinorelbine, Vinorelbine tartrate, VLB, VP-16, Vumon, Xeloda, Zanosar, Zevalin, Zinecard, Zoladex, Zoledronic acid, Zometa, Gliadel wafer, Glivec, GM-CSF, Goserelin, granulocyte colony stimulating factor, Halotestin, Herceptin, Hexadrol, Hexalen, Hexamethylmelamine, HMM, Hycamtin, Hydrea, Hydrocort Acetate, Hydrocortisone, Hydrocortisone sodium phosphate, Hydrocortisone sodium succinate, Hydrocortone phosphate, Hydroxyurea, Ibritumomab, Ibritumomab Tiuxetan, Idamycin, Idarubicin, Ifex, IFN-alpha, Ifosfamide, IL 2, IL-11, Imatinib mesylate, Imidazole Carboxamide, Interferon alfa, Interferon Alfa-2b (PEG conjugate), Interleukin 2, Interleukin-11, Intron A (interferon alfa-2b), Leucovorin, Leukeran, Leukine, Leuprolide, Leurocristine, Leustatin, Liposomal Ara-C, Liquid Pred, Lomustine, L-PAM, L-Sarcolysin, Meticorten, Mitomycin, Mitomycin-C, Mitoxantrone, M-Prednisol, MTC, MTX, Mustargen, Mustine, Mutamycin, Myleran, Iressa, Irinotecan, Isotretinoin, Kidrolase, Lanacort, L-asparaginase, LCR, FAM-HYD-1, Marizomib (NPI-0052), Lenalidomide, Carfilzomib, Panobinostat, Quisinostat, Selinexor, and Oprozomib as well as any agent listed in FIG. 7. The anticancer agent can also include biopharmaceuticals such as, for example, antibodies.

64. In some examples, the active agent can comprise a combination of active agents. In some examples, the active agent can comprise melphalan, bortezomib, FAM-HYD-1, Marizomib (NPI-0052), Carfilzomib, Cytoxan, Dexamethasone, Daratumumab, Doxorubicin, Thalidomide, Lenalidomide, Oprozomib, Panobinostat, Pomalidomide, Quisinostat, Selinexor, venetoclax, or a combination thereof, such as, for example, carfilzomib/panobinostat; daratumumab/bortezomib; carfilzomib/dexamethasone; carfilzomib/pomalidomide; bortezomib/dexamethasone, selinexor/doxorubicin, dexamethasone/venetoclax, and selinexor/dexamethasone. Additional agent combinations include, but are not limited to bortezomib and 113; bortezomib and adavosertib; bortezomib and AZ-628; bortezomib and CGP-60474; bortezomib and CP-724714; bortezomib and CPD22; BDa, bortezomib and dabrafenib; bortezomib and JNK-IN-8; bortezomib and lenalidomide; bortezomib and MARK-INHIBITOR; bortezomib and melphalan; bortezomib and NU-7441; bortezomib and R406; bortezomib and silmitasertib; bortezomib and TAI-1; carfilzomib and adavosertib; carfilzomib and dabrafenib; carfilzomib and dinaciclib; carfilzomib and GDC-0980; carfilzomib and JNK-IN-8; carfilzomib and lenalidomide; carfilzomib and MARK-INHIBITOR; carfilzomib and melphalan; carfilzomib and panobinostat; carfilzomib and pomalidomide; carfilzomib and R406; carfilzomib and volasertib; daratumumab and bortezomib; daratumumab and carfilzomib; daratumumab and ixazomib; daratumumab and lenalidomide; defactinib and melphalan; dexamethasone and ABT-199; dexamethasone and lenalidomide; dexamethasone and pomalidomide; ixazomib and ABT-199; ixazomib and motesanib; selinexor and 111; selinexor and alisertib; selinexor and dabrafenib; selinexor and doxorubicin; melphalan and 113; melphalan and panobinostat; melphalan and VS4718; melphalan and ONX; panobinostat and ABT-199; panobinostat and dexamethasone; and pomalidomide and pyrvinium; or any other combination shown in Table 1. Bortezomib, carfilzomib, and oprozomib are proteasome inhibitors, whereas melphalan is an alkylating agent. They are approved for the treatment of multiple myeloma, as well as other diseases. FAM-HYD-1 is a conjugate of the fluorescent molecule fluorescein (FAM) and the 1.5 kDa peptide HYD-1, an experimental drug with direct toxicity to MM cells (Nair R R, Emmons M F, et al. Mol Cancer Ther 2009; 8:2441-51). Panobinostat and Quisinostat are experimental histone deacetylase (HDAC) inhibitors in clinical trials for treatment of multiple myeloma patients. Selinexor is a nuclear export inhibitor also in clinical trials for treatment of multiple myeloma.

65. Contacting the cells with the active agent can be accomplished by any suitable method and technique presently or prospectively known to those skilled in the art. Administration of the active agent can be a single administration, or at continuous or distinct intervals as can be readily determined by a person skilled in the art.

66. In some examples, the first optical signal, the second optical signal, or a combination thereof involves any optical microscopy illumination techniques suitable to detect cell membrane activity, such as a bright field illumination, dark field illumination, fluorescence microscopy, and phase contrast illumination.

67. Cell membrane motion can comprise, for example, observable changes in the size and/or morphology of the cell membrane (e.g., cell membrane motion does not comprise translational motion of the cell). In some examples, the absence of cell membrane motion can indicate cell death.

68. In some examples, the cells of the method are obtained by collecting a sample from the subject and then isolating the cells from the sample. As an example, the sample can comprise a bone marrow aspirate where the cells are hematological cancer cells isolated from the aspirate, e.g., by flow cytometry using a cell surface cancer marker.

69. In some examples, the method can further comprise collecting parameters from the viability observations to generate a multi-parameter model that summarizes the response of a cancer in a subject to the active agent. These parameters can include, for example, drug concentration, exposure time, IC50, EC50, and drug free doubling time, as well as clinical information from the patient, such as previous response to drugs and rate of tumor regrowth as measured by surrogate measurements such as blood or urine para-proteins. Computational methods, such as those disclosed herein, may be parameterized by data from the disclosed method and used to estimate response to treatment with the drug being tested.

70. Also disclosed herein are methods for predicting a response of a subject to treatment with an active agent. The methods can comprise first preparing a three-dimensional dose-response curve by assessing the viability of cells from the subject in response to the active agent at a plurality of time points at a plurality of dosages. The method can then involve generating a multi-parameter model that summarizes the three-dimensional dose-response curve. The multi-parameter model can then be used to calculate the rate of accumulation of damage in the cells due to the active agent and the active agent-induced cell death due to the accumulated damage.

In some embodiments, the number of distinct populations (e.g., in terms of sensitivity to the active agent) in the cells is a covariate in the multi-parameter model, so the method can involve determining the number of populations. The rate of accumulation of damage in the cells and the active agent-induced cell death due to the accumulated damage can then be extrapolated to predict a response of the subject to the active agent. For example, a three-dimensional dose-response curve based on 1, 2, 3, 4, 5, 6, 7, 8, 9, 10, 11, 12, 13, 14, 15, 16, 17, 18, 19, 20, 21, 22, 23, 24, 25, 28, 30, 32, 35, 36, 40, 42, 48 hours, 3, 4, 5, 6, 7, 8, 9, 10, 11, 12, 13, 14, 15, 16, 17, 18, 19, 20, 21, 22, 23, 24, 25, 26, 27, 28, 29, 30, 31, 35, 42, 49, 56, 60, 61, 62, or 90 days of viability data can be extrapolated to 1, 2, 3, 4, 5, 6, 7, 8, 9, 10, or more years of response by the subject. In some aspect, measurements can be obtained at least one time every 5, 10, 15, 20, 25, 30, 35, 40, 45, 50, 55, 60, 75, 90, 105, 120 minutes, 3, 4, 5, 6, 7, 8, 9, 10, 11, 12, 13, 14, 15, 16, 17 18, 19, 20, 21, 22, 23, or 24 hours.

71. In some examples, assessing the viability of the plurality of cells can comprise any of the methods described above.

72. In some examples, the methods disclosed herein can further comprise selecting a cancer treatment regimen for the subject based on predicted responses to 2, 3, 4, 5, 6, 7, 8, 9, 10, or more different active agents.

73. In some examples, the method can predict an initial response of the subject to the active agent. In some examples, the method can predict the chance of progression-free survival. In some examples, the method can predict the chance of developing environment-mediated resistance to the active agent. In some examples, the method can predict an effective dosing schedule of the active agent. In some examples, the method can predict an effective concentration of the active agent.

74. The methods disclosed herein can be carried out in whole or in part on one or more computing device. Therefore, also disclosed is a computer system comprising memory on which is stored instructions to perform the disclosed methods. Also disclosed herein are devices and modules within a device, wherein the device or module is configured to perform the disclosed methods. For example, the memory can contain instructions to receive optical signals from a device (e.g., imager), analyze the first optical signal and the second optical signal to detect cell membrane motion of the cells, and analyze the cell membrane motion to quantify the viability of the cells following contact with the active agent. In some examples, the memory can contain instructions to utilize a dose-response curve to develop a multi-parameter model, wherein the multi-parameter model describes the rate of accumulation of damage in the cells due to the active agent and the active agent-induced cell death due to the accumulated damage; utilize the multi-parameter model and the dose-response curve to determine the number of populations in the sample; and utilize the number of populations and the multi-parameter model to predict a response of the subject to the active agent.

75. FIG. 13 illustrates an example computing device upon which examples disclosed herein may be implemented. The computing device (160) can include a bus or other communication mechanism for communicating information among various components of the computing device (160). In its most basic configuration, computing device (160) typically includes at least one processing unit (212) (a processor) and system memory (214). Depending on the exact configuration and type of computing device, system memory (214) may be volatile (such as random access memory (RAM)), non-volatile (such as read-only memory (ROM), flash memory, etc.), or some combination of the two. This most basic configuration is illustrated in FIG. 13 by a dashed line (210). The processing unit (212) may be a standard programmable processor that performs arithmetic and logic operations necessary for operation of the computing device (160).

76. The computing device (160) can have additional features/functionality. For example, computing device (160) may include additional storage such as removable storage (216) and non-removable storage (218) including, but not limited to, magnetic or optical disks or tapes. The computing device (160) can also contain network connection(s) (224) that allow the device to communicate with other devices. The computing device (160) can also have input device(s) (222) such as a keyboard, mouse, touch screen, antenna or other systems. Output device(s) (220) such as a display, speakers, printer, etc. may also be included. The additional devices can be connected to the bus in order to facilitate communication of data among the components of the computing device (160).

77. The processing unit (212) can be configured to execute program code encoded in tangible, computer-readable media. Computer-readable media refers to any media that is capable of providing data that causes the computing device (160) (i.e., a machine) to operate in a particular fashion. Various computer-readable media can be utilized to provide instructions to the processing unit (212) for execution. Common forms of computer-readable media include, for example, magnetic media, optical media, physical media, memory chips or cartridges, a carrier wave, or any other medium from which a computer can read. Example computer-readable media can include, but is not limited to, volatile media, non-volatile media and transmission media. Volatile and non-volatile media can be implemented in any method or technology for storage of information such as computer readable instructions, data structures, program modules or other data and common forms are discussed in detail below. Transmission media can include coaxial cables, copper wires and/or fiber optic cables, as well as acoustic or light waves, such as those generated during radio-wave and infra-red data communication. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

78. In an example implementation, the processing unit (212) can execute program code stored in the system memory (214). For example, the bus can carry data to the system memory (214), from which the processing unit (212) receives and executes instructions. The data received by the system memory (214) can optionally be stored on the removable storage (216) or the non-removable storage (218) before or after execution by the processing unit (212).

79. The computing device (160) typically includes a variety of computer-readable media. Computer-readable media can be any available media that can be accessed by device (160) and includes both volatile and non-volatile media, removable and non-removable media. Computer storage media include volatile and non-volatile, and removable and non-removable media implemented in any method or technology for storage of information such as computer readable instructions, data structures, program modules or other data. System memory (214), removable storage (216), and non-removable storage (218) are all examples of computer storage media. Computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by computing device (160). Any such computer storage media can be part of computing device (160).

80. It should be understood that the various techniques described herein can be implemented in connection with hardware or software or, where appropriate, with a combination thereof. Thus, the methods, systems, and associated signal processing of the presently disclosed subject matter, or certain aspects or portions thereof, can take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs can implement or utilize the processes described in connection with the presently disclosed subject matter, e.g., through the use of an application programming interface (API), reusable controls, or the like. Such programs can be implemented in a high level procedural or object-oriented programming language to communicate with a computer system. However, the program(s) can be implemented in assembly or machine language, if desired. In any case, the language can be a compiled or interpreted language and it may be combined with hardware implementations.

81. Also disclosed herein are methods for selecting a cancer treatment regimen for a subject. The method can involve an in vitro microfluidic dose-response assay of a sample from a cancer of a subject to identify the response to one or more anticancer agent(s), such as a chemotherapeutic agent, compared to a control, another agent, or the additive effect of each agent assuming independent effect to determine the synergistic or antagonistic effect of any combination of drugs. The assay can involve the use of an observation chamber for visualizing cancer cells from the sample during the method. In some embodiments, the chemotherapeutic agent is diffused from one reservoir of a microfluidic chamber to the other thereby creating a stable gradient across the observation chamber. In some embodiments, cells are imaged continuously, allowing for the effect of time to be assessed. The assay can involve the use of concentration of a drug at a particular time or over time, a measure of drug induced damage, the repair rate of the cancer cell to detect the effect of each drug on the sample. The assay can utilize a synergy augmented model (SAM) to detect the synergistic effect of the drug combination relative to additive or independent drug applications.

82. In some examples, the method can further involve identifying cell death induced by the drug. Typical membrane-impermeable probes for detection of cell death, such as EthD-1, present a significant variation in the time for fluorescence acquisition after death in cell lines or patient samples. To avoid this confounding effect, disclosed is an approach that identifies cell death based of motion of the membrane. In some embodiments, the identification of cell death comprises: (a) collecting a first bright field image of a cancer cell at a first time; (b) collecting a second bright field image of a cancer cell at a second time; (c) applying an algorithm to the first and second images to identify the presence or absence of cell membrane motion; wherein the absence of cell membrane motion indicates cell death. Typical cell viability assays are often destructive or cytotoxic, if carried for long periods of time, limiting the information acquired in the temporal dimension. In the disclosed system and method, cancer cells, stroma and matrix do not have to be separated, and no cytotoxic agents have to be used to determine cell viability, thus allowing longitudinal studies of drug activity without interfering with the microenvironment. In some embodiments, only bright field imaging is used, thereby eliminating any toxicity from viability markers.

83. In some embodiments, the in vitro microfluidic dose-response assay comprises a combination of primary cancer cells from the sample, extracellular matrix, subject-derived stroma, and one or more growth factors. The extracellular matrix and stroma are components of chemoresistance in many tumors. However, the inclusion of these elements significantly increases the complexity of dose response assays, often requiring the separation between cancer and stromal cells, by matrix digestion and/or flow sorting (Misund K, Baranowska K A, et al. J Biomol Screen 2013; 18:637-46). Cell adhesion mediated drug resistance (CAMDR) is believed to be a cause of minimal residual disease in multiple myeloma (Meads M B, Gatenby R A, Dalton W S. Nat Rev Cancer 2009; 9:665-74). In some embodiments, the assay allows for assessment of environment-mediated drug resistance.

84. In cancers such as MM, where a few million cells are obtainable per patient biopsy, it is important to minimize the number of cells per experimental condition. In some embodiments, less than 20,000 cancer cells are used in the assay described herein (for example, less than 20,000; 15,000; 10,000; 5,000 or 2,000 cells). In some embodiments, more than 1,000 cells are used in the assay (for example, at least 1,000; 2,000; 3,000; 4,000; 5,000; 6,000, 7,000; 8,000; 9,000; or 10,000 cells). In some embodiments, 1,000-10,000 cells are used in the assay (for example, at least 1,000; 2,000; 3,000; 4,000; 5,000; 6,000, 7,000; 8,000; 9,000 or 10,000 cells).

85. The disclosed system and method can further involve collecting or estimating parameters from the assay to generate a multi-parameter model that summarized the response of the subject to the drug treatment. These parameters include, for example, drug concentration, exposure time, IC50, EC50, and drug free doubling time. Computational methods, such as those disclosed herein, may be parameterized by data from the disclosed method and used to estimate response to treatment with the drug being tested.

86. The disclosed system and method can be used to select a cancer treatment regimen for the subject based on the results of the multi-parameter model. In some embodiments, the integration between in vitro and in silico computational models allows for assessment of initial response to a drug. In some embodiments, the integration between in vitro and computational models allows for assessment of the progression-free survival.

87. In some embodiments, the cancer is a hematological malignancy. In some embodiments, the sample is a bone marrow aspiration. In some embodiments, the cancer is multiple myeloma.

88. The disclosed method may be used to identify drug candidates for any cancer type or subtype. A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer.

89. In some embodiments, combinations of drugs are tested. In some cases, the dosing schedule of a combination of drugs is tested. In some embodiments, the heterogeneity of drug response is assessed. In some embodiments, the drug comprises melphalan, bortezomib, FAM-HYD-1 or combinations thereof.

### C. EXAMPLES

90. The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: Pharmacodynamic Model for Clinical Synergy

91. In the field of clinical pharmacology, there are multiple definitions for drug additivity. Bliss, for example, assumes statistical independence in the action of the drugs in a given combination, whereas Loewe defines it as a scenario where the reduction in dose of one drug proportionally complements the dose reduction achieved due to the second drug. Herein, synergy is defined as a benefit over an additive response as it better matches the reality of the clinic: a physician will not reduce the dosing of drugs to achieve the same outcome, but would rather seek a tolerable combination with most improvement over its independent effects. The pursuit for synergistic drug combinations arises from the myriad of advantages of combination therapy, such as maximizing efficacy, reducing toxicity, and addressing interpatient variability, as well as delaying and overcoming innate or acquired resistance.

92. Innate or acquired resistance poses a major hurdle in effectively treating many cancers. Resistance to a drug can arise because of enhanced degradation of the drug, increased expression of the drug target, alteration of the target, clonal evolution, microenvironmental factors, or intratumoral heterogeneity. Thus, combination effect can be improved either by combining a drug that disrupts the mechanism of resistance of a second drug, or by combining drugs that target different subpopulations in the tumor.

93. In spite of the advantages seen in combination therapies, there are also adverse drug-drug interactions in patients. Furthermore, a combination proven to be statistically beneficial for a cohort of patients may not be the most promising option for each individual patient in the cohort, some patients can be further benefited by combinations tailored to their particularities and needs. However, absolute personalization of therapy would be impractical based solely on a patient's clinical history and clinical literature. Thus, therapeutic regimens can be screened using clinical decision support tools backed by experiments conducted using the patient's own biopsy samples to identify therapies that yield better outcomes and complement a physician's clinical acumen.

94. Combination effects in multiple myeloma (MM), a treatable yet incurable cancer of bone marrow-resident plasma cells, was investigated for several reasons. MM provides access to rich patient specimens from bone marrow biopsies. Due to inter- and intratumoral heterogeneity in MM, a priori knowledge of drug effects can markedly improve clinical outcomes. MM patients often respond well to initial therapy, but eventually relapse, and subsequent lines of therapy are characterized by ever-shortening responses followed by relapses, ultimately leading to multidrug resistance. Recent advances in clinical outcomes for MM patients are derived from the combination of novel agents. The common rationale is that these drugs potentiate each other's effects; however, there are no available tools to estimate clinical synergy (better than additive) or clinical benefit (better than either single agent) of combination therapy in MM or other malignancies.

95. Using tumor cells from MM patients, the Ex vivo Mathematical Malignancy Advisor (EMMA), a mathematical framework that estimates tumor-specific drug sensitivity from patient-derived primary MM cells in an ex vivo reconstruction of the bone marrow microenvironment, was developed. EMMA relies on a drug-agnostic mechanistic model comprised of a dose-effect relationship at the pharmacodynamic level and a cumulative effect-response relationship to estimate the percent tumor burden within a clinically actionable time frame (6 days). However, in its previous form, EMMA lacked the ability to capture combination effects for combination therapies. Instead, it assumed the effect to be additive, as defined by the Bliss independence model. To address this, a synergy-augmented model (SAM) was developed, which captures interactions between drugs ex vivo and translates combination effects from fixed ex vivo drug concentrations to clinically relevant time-varying concentrations modeled from pharmacokinetic data. By incorporating SAM into the high-throughput testing of drugs on fresh primary MM cells, it was shown how this new drug-agnostic synergy-modeling framework can serve as an effective tool in identifying the most viable combination at a given point in each patient's treatment history.

a) Results
(1) Modeling Tumor-Specific Single-Agent Sensitivity Ex Vivo Using EMMA

96. EMMA is a mathematical modeling framework powered by a high-throughput novel ex vivo assay, where primary MM cells treated with 31 drugs/combinations are imaged every 30 minutes for up to 6 days in an ex vivo reconstruction of the tumor microenvironment. At the center of this mathematical framework rests the concept that drug-induced damage drives the rate of cell death when the damage exceeds a tumor-specific threshold. FIG. 1A depicts drug-induced decrease in cell viability (in percent, normalized by viability at time=0 hours) for one patient's (patient 210's) primary MM cells treated ex vivo with 0.05 μM of the proteasome inhibitor carfilzomib for an interval of 120 hours. Linear decay and Michaelis-Menten models can fit the late dynamics of drug-induced cell killing, but, unlike EMMA, they are unable to describe an approximate 30-hour delay between start of treatment and initiation of cell death. This delay is further magnified at lower concentrations, where increasing intervals of drug exposure are required to initiate cell death. This limitation results from a direct functional dependence of cell death rates on drug concentration in these models. EMMA, on the other hand, is a second-order model that requires accumulation of drug-induced damage beyond a certain threshold before the observed cell death can occur.

97. The dose-effect relationship for a single agent is governed by a reversible reaction kinetic equation (FIG. 1B) where: R(t) is the concentration of the drug at time t; β(t) represents drug-induced accumulated damage, or the “effect” in the dose-effect relationship; κ is the tumor-specific cell damage reduction, or repair rate; and h is an empirical exponent that couples the stoichiometry of drug concentration to the damage effect in the cell. Drug-induced damage (β) accumulates with drug exposure and decreases with cell repair. Cell death only initiates after β crosses a tumor-specific threshold (τ) and proceeds at a rate governed by a sigmoidal function.
(2) Modeling Patient's Clinical Response to Combination Therapy Assuming Additivity

98. EMMA's clinical predictions have been shown to be accurate and reproducible in a cohort of 52 MM patients treated with different combination regimens, assuming additivity. In order to simulate the clinical responses of MM patients, a tumor growth model (FIG. 1C) was included. Briefly, it is a doubling time equation, where LI is the labeling index, or percentage of replicating cells, assumed to vary between 1% and 3%, and p(t) is the tumor burden at time t, in hours. Intratumoral heterogeneity of sensitivity to single agents was estimated by fitting ex vivo drug sensitivity data to models of increasing complexity, where the entire tumor was described by one or two subpopulations, each subpopulation being either clonal or represented by a normal distribution. Akaike information criterion (AIC) was used to choose the model that best represents the data. Tumor drug-specific parameters of the best-ranked AIC model were coupled with pharmacokinetic data from phase I clinical trials to simulate patient/drug-specific clinical response. EMMA-based clinical predictions of combination regimens assumed additivity. Here, this framework is advanced to model the combination effect between agents.
(3) Modeling Tumor-Specific Two-Drug Combination Interactions Ex Vivo Using SAM

99. FIG. 1D depicts the ex vivo drug response of primary MM cells of a patient (patient 290) to the combination of 0.05 μM carfilzomib and 0.05 μM panobinostat (solid blue line), as well as single-agent responses (carfilzomib in red and panobinostat in green). The “additive” response (dashed blue line) was computed as a pointwise product of the fractional viability of the 2 single agents, as per the Bliss independence model, assuming statistical independence between the effects of each drug. In this example, the actual combination of the 2 drugs is more effective in killing MM cells than the predicted additive effect, and thus is considered synergistic. The combination effect, however, is dynamic, varying with exposure time, drug concentrations, and the tumor cells being tested. Thus, similar to the modeling approach employed to estimate tumor-specific parameters governing the single-agent response, a model for the combination effect accounting for these different variables was developed (FIG. 1E).

100. Assume a sample is simultaneously treated with 2 drugs: A and B. The EMMA framework was extended to account for the two-way combination effect by incorporating into the pharmacodynamic equation of the first drug (Drug A) a term that represents the effect of the second drug (Drug B) on the first: effect of Drug B on Drug A. Conversely, the same was done for Drug B. The action of Drug A (R_{A} in Equation (1)) as a single agent causes the damage β_{A} in Equation (1),

$${β}_{A} ( t ) = {\int }_{0}^{t} exp ( - {κ}_{A} ( t - \tilde{t} ) ) {{R}_{A} ( \tilde{t} )}^{{h}_{A}} d \tilde{t} , \qquad ( 1 )$$

where κ_{A} and h_{A} are estimated from single-agent EMMA model for Drug A. Similarly, Drug B's single-agent damage, β_{B}, is described by Equation (2):

$${β}_{B} ( t ) = {\int }_{0}^{t} exp ( - {κ}_{B} ( t - \tilde{t} ) ) {{R}_{B} ( \tilde{t} )}^{{h}_{B}} d \tilde{t} . \qquad ( 2 )$$

Equations (1) and (2) are closed-form solutions of the single-agent pharmacodynamic equation shown in FIG. 1B. When combined, in addition to the effects of the 2 drugs acting alone, there are two combination effects due to the interaction of the 2 drugs. For example, the effect of Drug B on Drug A, β_{BA}, is described in Equation (3),

$${β}_{B A} ( t ) = {\int }_{0}^{t} exp ( - {κ}_{A} ( t - \tilde{t} ) ) {f}_{B A} ( {{R}_{A} ( \tilde{t} )}^{{h}_{A}} , {{R}_{B} ( \tilde{t} )}^{{h}_{B}} ; {λ}_{B A} ) d \tilde{t} , \qquad ( 3 )$$

where λ_{BA} represents a parameter set and ƒ_{BA} is the function that defines the combination effect between Drug A and Drug B such that ƒ_{BA} (R_{A}^{h}^{_{A}},R_{B}^{h}^{_{B}})=0, ∀ R_{A}=0, or R_{B}=0. The simplest mathematical expression that satisfies the above condition is the bilinear function ƒ_{BA}(R_{A}^{h}^{_{A}},R_{B}^{h}^{_{B}})=γ_{BA}R_{A}^{h}^{_{A}},R_{B}^{h}^{_{B}}, where γ_{BA} is an undetermined coefficient to be estimated using the fixed-ratio combination ex vivo sensitivity data. Similarly, the effect of Drug A on Drug B, β_{AB}, is given by Equation (4):

$${β}_{A B} ( t ) = {\int }_{0}^{t} exp ( - {κ}_{B} ( t - \tilde{t} ) ) {f}_{A B} ( {{R}_{A} ( \tilde{t} )}^{{h}_{A}} , {{R}_{B} ( \tilde{t} )}^{{h}_{B}} ; {λ}_{A B} ) d \tilde{t} . \qquad ( 4 )$$

Thus, damage caused by R_{A} in the presence of R_{B} is β_{A/A+B}=β_{A}+β_{BA} and damage caused by R_{B} in the presence of R_{A} is β_{B/A+B}=β_{B}+β_{AB}. Estimated from the single-agent models and fixed-ratio ex vivo combination, damages β_{A/A+B} and β_{B/A+B} accumulate over time, and cell death initiates when either exceeds the tumor-specific thresholds TA or TB, respectively. The accumulated damages β_{A} and β_{B} result in changes in the viability, given by dp_{A}(t)/dt and dp-_{B}(t)/dt, respectively. Viability of the two-drug combination, p(t), is given by the product between p_{A}(t) and p_{B}(t), assuming statistical independence, since the interaction between R_{A} and R_{B} was already accounted for by β_{BA} and β_{AB}. This modeling framework, capturing the two-way combination effect from patient-specific ex vivo response measurements, is SAM.

101. This first version of SAM assumes monotonicity of the combination effects between the 2 drugs. However, combination studies often show that a non-monotonic relationship may exist where particular concentrations, or “sweet spots,” of either or both drugs yields the maximum combination effect. In order to allow the model to account for such an effect, a second-order polynomial was chosen to describe the functional dependence between the combination effect (ƒ_{BA}) and the concentration of the aiding drug (R_{B}) which required additional parameters (ε_{BA}) for each of the 2 drugs (Equation (5)). In every two-drug combination in this work, both monotonic and non-monotonic SAM models were calculated and applied a modified AIC (derivation of AIC for a composite experiment) to choose the simplest model for a composite experiment that best fits the data over noise (additive model, monotonic, or non-monotonic SAM).

ƒⱼᵢ(Rᵢ^{h}^{ᵢ},Rⱼ^{h}^{ⱼ})=Rᵢ^{h}^{ᵢ}(γⱼᵢ+òⱼᵢRⱼ^{h}^{ⱼ})Rⱼ^{h}^{ⱼ},∀(i,j)={(A,B),(B,A)} (5)

(4) Validation of SAM Ex Vivo

102. Herein is tested whether SAM can be parameterized exclusively by single-agent EMMA parameters and fixed-ratio combination ex vivo data by comparing SAM's model predictions with ex vivo results from an actual drug combination matrix (checkerboard assay). In every experiment, each drug was tested in 5 different concentrations following a 1:3 dilution, in duplicates, except for the combination matrix, where quadruplicates were used. FIGS. 2A-C depict the ex vivo response of primary MM cells (derived from patient 385) to single-agent carfilzomib and panobinostat, as well as to the combination of both drugs at a fixed concentration-ratio (1:1), respectively. The results from the combination matrix are presented in FIG. 2D, where the plots highlighted in red are the fixed-ratio concentrations used for estimating SAM parameters. Each plot in FIG. 2D depicts the data points for measured cell viability (colored dots for different replicates), a smoothed (locally weighted scatter plot smoothing [LOWESS] algorithm) curve of the ex vivo cell viability data (dashed black line), and the SAM model prediction (solid line).

103. FIG. 2E depicts Pearson's correlation coefficient (r) between SAM's model predictions and smoothed ex vivo response for each of the 25 two-drug combination concentration pairs, showing high linear correlation between model predictions and experimental measurements (r>0.93). Similarly, FIG. 2F shows the angle of the slope (α) of the linear regression, ranging between 45° (green) and 50° (yellow), where an α value of 45° and r=1 represent a perfect linear correlation. Additional comparisons with a second sample (patient 390) and 2 other pairs of drugs (carfilzomib/dexamethasone and carfilzomib/panobinostat) are provided as FIGS. 3-5.

(5) SAM as a Tool to Study Combination Effect of Drugs in Primary Samples Ex Vivo

104. The Loewe Combination Index (CI) of 130 drug combinations tested was calculated with primary MM samples in EMMA, according to Chou-Talalay's method using the median lethal dose (LD50, defined as the drug concentration that reduces cell viability to 50% of initial measurement) and 96-hour time point as metrics. By definition, this method requires that both drugs reach the cell-kill effect (LD50) at the time point of CI calculation (96 hours), which reduced the number of drug combinations with CI down to 62. FIG. 6A depicts the 10 drug combinations with the lowest (most synergistic) and highest (most antagonistic) values of median CI (the entire set is available in FIG. 7). The wide range of ex vivo cell-kill effects of MM-relevant classes of drugs significantly limited the application of this method; while proteasome inhibitors (e.g., bortezomib) or chemotherapeutic agents (e.g., melphalan) can induce LD50 in less than 48 hours; immunomodulators (e.g., pomalidomide), steroids (e.g., dexamethasone), or immunologics (e.g., daratumumab) required over 96 hours to reduce cell viability to 30% of initial measurements, even at maximum solubility levels. In addition, because of inherent interpatient tumor heterogeneity, the calculation of the combination effect between 2 drugs can be performed using group statistics in a cohort of samples.

105. FIG. 6B depicts a novel combination effect analysis to address these limitations. In this volcano plot, the horizontal axis represents the log₂ fold-change in median LD50 between the actual ex vivo two-drug combination, and the theoretically computed additive response, which was calculated assuming statistical independence between the cell-kill effects of both drugs: the single-agent ex vivo dose-time-response surfaces of each drug in the combination (e.g., FIG. 2A-B) were multiplied pointwise to generate the theoretical additive response curve, from which LD50 was computed at 96 hours. The vertical axis represents the −log₁₀ P value for a two-tailed paired t-test conducted between the theoretical additive and the actual LD50-at-96-hours values for all samples tested with the combination. FIG. 6C exemplifies this test for the combination of carfilzomib and panobinostat, a combination that is consistently synergistic, as evidenced by the overwhelming number of samples where the LD50 of the combination was lower than predicted by additivity. Thus, to use this approach, it is sufficient that the actual ex vivo combination, and theoretical additive combination, reached LD50, instead of both single agents. In addition, FIG. 6B provides a statistical measurement of magnitude and heterogeneity of the combination effect in the group of samples.

106. This combination effect analysis was further extended by introducing a second measure of ex vivo drug resistance: the average area under the curve (AUC) of the 5 concentrations from the beginning of the experiment until the final time point (e.g., 96 hours). The benefit of this second metric is that it is not bound to an arbitrary minimum cell-kill effect, thus allowing direct comparisons among drugs and combinations with significantly different cell-kill dynamics. FIG. 6D-E reflects the same analyses as FIG. 6B-C, except that they compare the actual versus theoretical additive values of the AUC.

107. Importantly, the most synergistic ex vivo combinations identified by this approach are part of clinical combination regimens in MM: carfilzomib/panobinostat; daratumumab/bortezomib; carfilzomib/dexamethasone; carfilzomib/pomalidomide; and selinexor/dexamethasone, recently approved to treat refractory MM.
(6) Estimation of clinical synergy

108. Summary metrics such as LD50 and AUC, while useful for preclinical studies, are unable to account for the complex pharmacokinetic/pharmacodynamic interactions of actual clinical regimens. Since the magnitude of drug combination effects vary with exposure time and drug concentrations, it is imperative to use models that can analyze combination effects in clinically relevant concentrations while also accounting for drug-specific pharmacokinetics. To this effect, patient-specific SAM models were parameterized with drug-specific phase I pharmacokinetic data to estimate magnitude and interpatient heterogeneity of the clinical combination effects of drug regimens in MM. FIG. 8 contains 4 synergy maps, which define regions of synergy and antagonism of 2 drugs (carfilzomib/dexamethasone) as a function of drug concentrations and exposure time in primary MM samples ex vivo. FIG. 8A presents the synergy map for one MM patient's (patient 135's) primary cell ex vivo response to carfilzomib and dexamethasone, where red-yellow (hot) regions denote synergy and blue-cyan (cold) represent antagonism. The combination effects for each point in the 3D space were calculated as the difference between the viability of the actual ex vivo combination, as predicted by SAM, and the theoretical additive viability, computed (FIG. 6). Also part of the synergy map is a pharmacokinetic trajectory (black ribbon), representing the varying concentrations of both drugs during the first 96 hours of the combination regimen. FIG. 8B represents the simulation of treatment of the same patient (patient 135) with either of the single agents, as well as the theoretical additive combination and the clinical prediction, based on SAM data. In this simulation, the patient is resistant to carfilzomib, but sensitive to dexamethasone, reaching approximately 50% tumor reduction after 3 months of treatment, based on the additive model. However, when the combination effect is considered, the predicted clinical response is 75% tumor burden reduction, and thus clinically synergistic.

109. In this particular patient/drug combination, the pharmacokinetic trajectory of treatment was confined to synergistic or additive regions (FIG. 8A), which explains the clinical synergy of the model predictions. However, when the pharmacokinetic trajectory crosses regions of both synergy and antagonism (FIG. 8C) or mainly additivity (FIG. 8E), the resulting clinical effect is additive (FIGS. 8D and 8F), and incidentally, when the pharmacokinetic trajectory is located in regions of antagonism (FIG. 8G), the predicted clinical outcome is antagonistic (FIG. 8H).
(7) SAM as a Tool to Estimate the Clinical Combination Effect and Clinical Benefit of Drug Combinations

110. Patient-specific SAM parameters, estimated by fitting ex vivo drug/combination sensitivity data for 203 patients, were coupled with pharmacokinetic data from phase I clinical trials to estimate the combination effect and clinical benefit of 46 (out of 130) two-drug combinations (FIG. 9), which have publicly available pharmacokinetic data. In this context, the combination effect is considered synergistic if the minimum tumor burden, as estimated by SAM, is lower than the theoretical additive (as described in FIG. 8) and is considered antagonistic if the opposite is true. Clinical benefit was defined as the improvement in clinical response of the SAM-estimated combination compared to the clinical response of the best single agent.

111. FIG. 9A's volcano plot depicts the clinical drug combination effect, showing on the vertical axis the −log₁₀ (P value) from the two-tailed paired t-test between theoretical additive and SAM-estimated best response predictions, the horizontal axis represents the median percent tumor burden change between theoretical additive and SAM-estimated combination. Among the 46 combinations tested, 4 were classified as clinically synergistic: daratumumab/bortezomib, carfilzomib/panobinostat, selinexor/dexamethasone, and selinexor/doxorubicin. FIGS. 9C-F represent these 4 combinations, where the first and fourth columns show simulated best responses for each single agent, the second column represents theoretical additive best response, and the third column represents the SAM-calculated best response of the combination. The best response for a therapeutic option is defined as the lowest tumor burden observed over a treatment period (90 days). The left vertical axis represents the tumor burden reduction from the start of treatment (with 0% corresponding to no response and 100% corresponding to total tumor eradication). The right vertical axis represents tumor burden reduction according to International Myeloma Working Group's classification of the depth of response. The values of the 4 columns corresponding to each patient are linked by a dashed line, lines for patients with synergistic combinations are red and antagonistic combinations are blue. The solid red lines in FIGS. 9C-F highlight the most synergistic patient within each drug combination. The synergy maps for each of these patients are shown in FIGS. 9G-J and confirm that the pharmacokinetic trajectories of these drug combinations are confined to regions of synergy in all 4 patients. Conversely, FIG. 10 highlights in solid blue lines the most antagonistic patient responses for each of the 4 drug combinations, and their corresponding synergy maps confirm that the pharmacokinetic trajectories are confined by regions of antagonism.

112. Clinical trials, however, do not assess combination effect, but clinical benefits. For example, phase III trials quantify the clinical benefit of a new agent by treating patients in one arm with the standard of care therapy, while patients in the experimental arm are treated with a combination of the standard of care and the new agent. A trial is considered successful if, in addition to meeting safety and toxicity standards, the experimental arm patients have a better outcome than the standard of care arm. FIG. 9B reflects this concept in a volcano plot where SAM-predicted combination clinical responses are compared to the predicted responses of the more efficacious of the 2 single agents. Similar to FIG. 9A, in FIG. 9B a paired t-test was used to compute the P value (vertical axis) and the difference in the medians (horizontal axis) for each drug pair. In addition to the 4 clinically synergistic drug combinations identified in FIG. 9A, five new ones were predicted to perform better than either single agent did independently: carfilzomib/dexamethasone, bortezomib/dexamethasone, carfilzomib/pomalidomide, bortezomib/pomalidomide, and dexamethasone/venetoclax.

b) Discussion

113. Multidrug combination therapies have been instrumental in improving efficacy in the treatment of MM. However, inter- and intrapatient heterogeneity of tumor sensitivity to single agents leads to variability in the combination effects of therapy. Described herein is a high-throughput assay designed to test the chemosensitivity of primary MM cells cultured in an ex vivo reconstruction of the bone marrow microenvironment, and, ultimately, to predict clinical response to therapies. This model (EMMA), however, relies solely on additive effects of individual agents. Here, this platform was extended. Using fixed-ratio ex vivo two-drug combination response data with a high sampling rate, a novel pharmacodynamic model (SAM) capable of capturing the two-way synergistic effect found in two-drug combinations was fit. This improvement further increases the original model's ability to estimate clinical response by accounting for the synergistic effects of combination regimens.

114. As a preliminary validation of SAM's ability to estimate combination effect, a checkerboard assay was used to measure the ex vivo response of two primary samples to 3 pairs of drug combinations in a 5×5 concentration combination matrix. It was shown herein that, when parameterized with the single agent and fixed-ratio combination, SAM accurately estimates ex vivo drug response to other drug concentration combinations, confirming that this reduced dataset is sufficient to parameterize this combination effect model.

115. The CI for was computed a comprehensive panel of two-drug combinations tested in a cohort of primary MM samples, and it was described how SAM can extend this well-established model of synergy to classes of drugs with significant differences in potency as well as account for intertumor heterogeneity. In this process, a list of ex vivo synergistic drugs were identified, including a number of combinations that are currently approved for MM therapy.

116. In order to investigate synergy in clinical regimens, patient-specific SAM models were used to simulate clinical response to combination regimens by parameterizing these models with clinical pharmacokinetic data. Using a graphical representation of combination effect as a function of drug concentrations and exposure time, it was demonstrated how the pharmacokinetic trajectory of drug concentrations crosses regions of synergy and antagonism during a cycle of a regimen, defining if the clinical response is synergistic or antagonistic.

117. An analysis of 46 drug pairs with pharmacokinetic data from clinical trials revealed 4 clinically synergistic combinations: daratumumab/bortezomib, carfilzomib/panobinostat, selinexor/dexamethasone, and selinexor/doxorubicin. This is consistent with a recent study that compared Kaplan-Meier curves from various phase II and phase III clinical trials in melanoma, ovarian, colorectal, pancreatic, and breast cancer patients treated with targeted therapies, immunotherapies, chemotherapies, etc., as single agents and combinations. The study revealed that the benefit of combinations over monotherapy is primarily due to independent drug action, reiterating that synergism observed preclinically did not necessarily translate into the clinic for most of the combinations. The authors showcased a study in PDX (patient-derived xenograft) patients, where testing 33 combinations across 6 tumor types revealed only four synergistic combinations.

118. Next, phase II/III clinical trials were simulated by assessing clinical benefit of a combination of 2 drugs over the best response of the more efficacious drug, identifying 5 additional combinations. These results were consistent with recent clinical studies in relapsed and refractory MM: the combination of daratumumab/bortezomib/dexamethasone was shown to be superior to the combination of bortezomib/dexamethasone in a phase III two-arm clinical trial. The efficacy of carfilzomib/panobinostat was studied in a phase I/II clinical trial setting and shown to be beneficial for relapsed/refractory MM patients. The combination of selinexor/dexamethasone has recently shown encouraging activity in a phase II trial involving highly refractory MM patients, leading to its FDA approval. Further, adding liposomal doxorubicin to the combination of selinexor/dexamethasone for relapsed and refractory MM patients is being currently studied as a phase I trial. Thus, the 4 combinations predicted to be clinically synergistic (FIG. 9A) have been shown to be at least clinically beneficial in clinical trials. In addition to these, the 5 other combinations identified as clinically beneficial have shown improved efficacy in multiagent clinical trials—carfilzomib/dexamethasone, bortezomib/dexamethasone, carfilzomib/pomalidomide, bortezomib/pomalidomide, and dexamethasone/venetoclax.

119. Of note, the results also indicate that, at least in this cohort, a number of drug pairs do not have synergistic activity (or even clinical benefit) across the majority of sample tested, but can be synergistic, or at least clinically beneficial, on a patient-by-patient bases. These observations further highlight the importance of accounting for interpatient heterogeneity and the need for personalized tools to improve clinical decisions as depicted in FIG. 9A, where a bird's-eye-view of 46 drug combinations can be ‘zoomed into’ single-drug pairs (FIG. 9C), and, finally, into specific pharmacokinetic/pharmacodynamic interactions in individual patient samples (FIG. 9G).

120. Combination therapy in MM typically involves combining two, three, or more drugs to maximize efficacy and time to relapse. The conclusions made from studying two-drug combinations can be extended to three-drug (or more) combinations by assuming that higher-order synergistic effects are negligible as shown in the literature. The approach used to compute three-drug combination response from two-drug responses is described in FIG. 11. The three-drug ex vivo combination response computed using this approach can be used to estimate AUC, as well as synergy, for any three-drug combination therapy received by a patient in the clinic. FIG. 15 depicts how these ex vivo measurements could be used to predict patients' clinical response. Using Receiver Operating Characteristic (ROC) curves, we show that ex vivo combination AUC serves as an excellent classifier of patients' clinical response between IMWG response stratifications of CR/VGPR (complete response/very good partial response), and partial response (PR) or worse with an area under the ROC curve of 0.9804 and a p-value of 0.0006 (for a t-test with the null hypothesis that the area under ROC is 0.5), while ex vivo synergy (ΔAUC Synergy), was the better classifier between PR/MR (minimal response/partial response) and SD/PD (stable disease/progressive disease) patients, with an area under the ROC curve of 0.8167 and a statistically significant p-value of 0.0452 (for a t-test with the null hypothesis that the area under ROC is 0.5). Furthermore, the proposed modeling framework can be used to modulate doses and schedules (within clinically viable limits) to maximize clinical synergy, and identify regimens that lead to significant improvement over the standard of care dosing for each patient.

c) Materials and Methods

(1) Ex Vivo Assay

121. An ex vivo assay was used to quantify the chemosensitivity of primary MM cells. Fresh bone marrow aspirate cells were enriched for CD138⁺ expression using Miltenyi (Bergisch Gladbach, Germany) 130-051-301 antibody-conjugated magnetic beads. MM cells (CD138⁺) were seeded in Corning (Corning, N.Y.) CellBIND 384 well plates with collagen I and previously established human-derived stroma to a total volume of 8 mL, containing approximately 4000 MM cells and 1000 stromal cells. Each well was filled with 80 μL of Roswell Park Memorial Institute (RPMI) 1640 media supplemented with fetal bovine serum (FBS, heat inactivated), penicillin/streptomycin, and patient-derived plasma (10%, freshly obtained from patient's own aspirate, filtered) and left overnight for adhesion of stroma. The next day, drugs were added using a robotic plate handler so that every drug/combination was tested at 5 (fixed concentration ratio, for combinations) concentrations (1:3 serial dilution) in two replicates. Negative controls (supplemented growth media with and without the vehicle control dimethyl sulfoxide [DMSO]) were included, as well as positive controls for each drug (cell line MM1.S at highest drug concentration). A plot of percent viability across time for negative control of Pt415, a 65 year-old female early relapsed/refractory MM patient, is shown in FIG. 14a. A marginal border effect amounting to a 10% increase in cellularity can be noticed in the plot during the first and last 6 hours of the experiment. This is an artefact of the image processing algorithm and hence, ex vivo responses of all drugged wells are normalized with primary MM control responses. A grouped bar plot shows a histogram of primary MM cellularity after 24, 48, 72, and 96 hours across 203 MM patients is presented in FIG. 14b. The histogram indicates the range of cellularity for majority of patients lies between 100 to 120 percent of initial value, while some specimens show a gradual decay upto 70%, others show a gradual increase upto 160% over 96 hours. These indicate that primary MM cells cultured ex vivo using the proposed approach survive for the duration of the experiment. Plates were placed in a motorized stage microscope (EVOS Auto FL, Life Technologies, Carlsbad, Calif.) equipped with an incubator and maintained at 5% CO₂ and 37° C. Each well was imaged every 30 minutes for a total duration of up to 6 days.

(2) Digital Image Analysis

122. A digital image analysis algorithm was implemented to determine changes in viability of each well longitudinally across the 96-hour interval. This algorithm computes differences in sequential images and identifies live cells with continuous membrane deformations resulting from their interaction with the surrounding extracellular matrix. These interactions cease upon cell death. By applying this operation to all 288 images acquired for each well, the effect of drugs as a function of concentration and exposure time was quantified nondestructively, and without the need to separate the stroma and myeloma.

(3) Model Fitting

123. There are two mathematical models that were fitted to the ex vivo data: EMMA for single agents and SAM for combinations. Both the models have distinct sets of equations, as described in FIG. 1. MATLAB's lsqcurvefit function from the Optimization toolbox was used to fit the ex vivo tumor burden measurements to these models, and parameters that govern tumor-drug/combination specific behavior were estimated. The optimization algorithm used for minimizing the sum of squares of the error between the model estimates and the actual data is called the trust-region-reflective method. This approach uses a quadratic form to restrict the step size of iterations when the initial guess is too far from the solution. This leads to a more reliable convergence to minima. The single-agent EMMA model involved four submodels that have a longitudinal variation in phenotypic heterogeneity, where the tumor is assumed to be a homogenous population, two homogeneous subpopulations, a normal distribution of subpopulations with varying thresholds to drug sensitivity, or two normal distributions of subpopulations. The convergence of the fitting was progressively improved for more complex models (models with a greater number of parameters) by using the converged solution of the less complex model as an initial guess. For example, the initial guess for one normal distribution of subpopulations can be the converged solution of the homogeneous population parameters with a negligible standard deviation. This allows lsqcurvefit to look for solutions in the neighborhood of the simpler model, thus providing insight into the need for complexity. Similarly, the SAM model has two submodels: monotonic SAM and non-monotonic SAM, where the converged solution of the monotonic SAM is used as the initial guess for the non-monotonic SAM. This approach improved the reliability of the convergence and ensured that the solutions to the more complex models are closer to the simpler ones.

(4) Model Selection

124. The single-agent EMMA model has 4 candidate submodels, each quantifying the phenotypic heterogeneity in a different manner. The choice of the best model cannot be purely based on the lowest sum of squares of residual (difference between the actual data and its corresponding model estimates), as noisy data seldom fits more complex models better than the simple ones due to the added degrees of freedom. The statistical model identification tool AIC was used to offset the goodness of fit with the complexity of the model (measured in terms of the number of parameters). Originally, AIC was developed to suit data obtained from a single experiment, but in the case of choosing between the 2 SAM models, the data comes from 3 different experiments: the 2 single agents' and the combinations' ex vivo assays. A modified AIC for a composite experiment was derived from first principles in derivation of AIC for a composite experiment, where the maximum log-likelihood function that minimizes the variance in the measurement noise was assumed to be for the composite experiment as a whole and not for individual experiments. This assumption facilitates the derivation of a maximum log-likelihood function to be used in the modified AIC that minimizes the variance in the measurement noise for the entire composite experiment.

(5) Derivation of AIC for a Composite Experiment

125. Let's assume that the model yⱼ=ƒⱼ(xⱼ|λⱼ) describes a process where, xⱼ∈□^{m} is the vector of independent variables, yⱼ∈□^{p} is the vector of dependent variables, and λⱼ∈□^{q}is the vector of undetermined parameters for the j^{th} experiment out of ‘n’ experiments. In order to estimate the undetermined parameters, ‘Nⱼ’ independent noisy measurements were made for the j^{th} experiment. The truth model for this experiment is

yⱼ=ƒⱼ(xⱼ|λⱼ)òⱼ

where, ò□N (0,σ²) is an independent and identically distributed (i.i.d) random variable vector describing Gaussian white noise with zero mean and variance σ² for the composite experiment. The measurement noise for the i^{th} observation in the composite experiment is

òᵢ=yⱼᵢ−ƒⱼ(xⱼᵢ|λⱼ),i=1, . . . ,N and j=1, . . . ,n

where,

$$N = {\sum }_{j = 1}^{n} {N}_{j}$$

and n is the number of experiments.
The probability density function for òᵢ is

$${l}_{i} ( {ò}_{i} \vert 0 , {σ}^{2} ) = \frac{1}{\sqrt{2 π {σ}^{2}}} exp ( - \frac{{{o}^{`}}_{i}^{2}}{2 {σ}^{2}} ) , i = 1 , … , N .$$

The likelihood function for ‘N’ observations is the product of probability density functions of individual observations (assuming each observation is made independently), it's given by

$$L ( {σ}^{2} \vert x , λ , y ) = {( \frac{1}{\sqrt{2 π {σ}^{2}}} )}^{N} exp ( - \frac{1}{2 {σ}^{2}} ( {\sum }_{j = 1}^{n} S S {R}_{j} ) ) where , {SSR}_{j} = {\sum }_{i = 1}^{{N}_{j}} {( {y}_{ji} - {f}_{j} ( {x}_{ji} \vert {λ}_{j} ) )}^{2} .$$

The natural logarithm of the likelihood function is

$$ln ( L ( {σ}^{2} \vert x , λ , y ) ) = - \frac{N}{2} ln ( 2 π ) - \frac{N}{2} ln ( {σ}^{2} ) - \frac{1}{2 {σ}^{2}} ( {\sum }_{j = 1}^{n} S S {R}_{j} ) .$$

Let \hat{σ}² be the variance that results in a maximum log-likelihood function value, then

$$\frac{d}{d {σ}^{2}} ln ( L ( {σ}^{2} \vert x , λ , y ) ) = - \frac{N}{2 {\hat{σ}}^{2}} + \frac{1}{2 {\hat{σ}}^{4}} ( {\sum }_{j = 1}^{n} S S {R}_{j} ) = 0. ⇒ {\hat{σ}}^{2} = \frac{{\sum }_{j = 1}^{n} {SSR}_{j}}{{\sum }_{j = 1}^{n} {N}_{j}} .$$

In other words, by minimizing the sum of squares of residuals of the individual experiments during data fitting we are also minimizing the variance of noise over the composite experiment. By substituting \hat{σ}² in the expression for the log-likelihood function,

$$ln ( L ( {\hat{σ}}^{2} \vert x , λ , y ) ) = - \frac{N}{2} ln ( 2 π ) - \frac{N}{2} ln ( {\hat{σ}}^{2} ) - \frac{1}{2 {\hat{σ}}^{2}} ( {\sum }_{j = 1}^{n} S S {R}_{j} )$$

$$⇒ ln ( L ( {\hat{σ}}^{2} \vert x , λ , y ) ) = - \frac{N}{2} ln ( \frac{{\sum }_{j = 1}^{n} S S {R}_{j}}{N} ) - \frac{N}{2} ln ( 2 π ) - \frac{N}{2}$$

$$- 2 ln ( L ( {\hat{σ}}^{2} \vert x , λ , y ) ) = N ln ( \frac{{\sum }_{j = 1}^{n} S S {R}_{j}}{N} ) + N ( ln ( 2 π ) + 1 )$$

$$AIC = - 2 ln ( L ( {\hat{σ}}^{2} \vert x , λ , y ) ) + 2 {\sum }_{j = 1}^{n} {k}_{j} = {\underbrace{( {\sum }_{j = 1}^{n} {N}_{j} ) ln ( \frac{{\sum }_{j = 1}^{n} {SSR}_{j}}{{\sum }_{j = 1}^{n} {N}_{j}} ) + 2 {\sum }_{j = 1}^{n} {k}_{j}}}_{Model Dependent} + {\underbrace{( {\sum }_{j = 1}^{n} {N}_{j} ) ( ln ( 2 π ) + 1}}_{Model Independent}$$

Therefore, for a composite experiment

$$AIC = ( {\sum }_{j = 1}^{n} {N}_{j} ) ln ( \frac{{\sum }_{j = 1}^{n} {SSR}_{j}}{{\sum }_{j = 1}^{n} {N}_{j}} ) + 2 {\sum }_{j = 1}^{n} {k}_{j} + ( {\sum }_{j = 1}^{n} {N}_{j} ) ( ln ( 2 π ) + 1 ) .$$

(6) Combination Matrix (Checkerboard Assay)

126. A combination matrix experiment was implemented on a 384-well plate using the approach described above, where CD138⁺ cells are seeded with collagen I and bone marrow stromal cells in media and the patient's plasma. The purpose of this assay was to show that the model predictions made using parameters estimated from fitting fixed concentration ratio ex vivo combination responses at various concentration magnitudes and ratios correlate well with experimental results. This was done by treating each well of the multi-well plate with a two-drug combination, where five serially (1:3) diluted concentrations of each drug were combined in 25 ways, best represented by a 5×5 matrix of concentration duplets. The concentration of one drug progresses from highest to lowest along rows and the second drug's concentration varies across columns, resulting in a constant concentration ratio along the diagonal. The five constant concentration ratio ex vivo responses (along the diagonal) are fit to SAM, and the model predictions at all the off-diagonal concentrations are compared with ex vivo responses.

(7) Primary Cancer Cells

127. The ex vivo responses of cancer cells from 203 MM patients were investigated; demographic data can be found in FIG. 12. Investigators obtained signed informed consent from all patients who were enrolled in the clinical trials MCC14745, MCC14690, and MCC18608 conducted at the H. Lee Moffitt Cancer Center and Research Institute, as approved by the Institutional Review Board. To this end, patient samples were used in accordance with the Declaration of Helsinki, International Ethical Guidelines for Biomedical Research Involving Human Subjects (CIOMS), Belmont Report, and U.S. Common Rule. The medical records were deidentified, and only the following clinically relevant information was reviewed: (A) the treatment administered (therapeutic agents, doses, and schedule) prior to biopsy, (B) cytogenetics, and (C) serum and urine electrophoresis results.

### D. References

Akaike H. New Look at Statistical-Model Identification. Ieee T Automat Contr 1974; Ac19(6):716-23 doi Doi 10.1109/Tac.1974.1100705.
Alexanian R, Dimopoulos M. The Treatment of Multiple Myeloma. New England Journal of Medicine 1994; 330(7):484-9 doi 10.1056/nejm199402173300709.
Al-Lazikani B, Banerji U, Workman P. Combinatorial drug therapy for cancer in the post-genomic era. Nat Biotechnol 2012; 30(7):679-91 doi 10.1038/nbt.2284.
Baz R C, Shain K H, Alsina M, Brayer J, Rashal T, Cooksey J L, et al. Phase I trial of the combination of selinexor (SEL), liposomal doxorubicin (DOX) and dexamethasone (Dex) for relapsed and refractory multiple myeloma (RRMM). J Clin Oncol 2016; 34(15_suppl):8013-doi 10.1200/JCO.2016.34.15_supp1.8013.
Berdeja J G, Gregory T B, Faber E, Matous J, Hart L, Mace J R, et al. A Phase I/II Study of the Combination of Panobinostat and Carfilzomib in Patients with Relapsed or Relapsed/Refractory Multiple Myeloma (MM): Final Analysis of Second Dose Expansion. Blood 2016; 128(22):4530-.
Bliss C I. The Toxicity of Poisons Applied Jointly. Annals of Applied Biology 1939; 26(3):585-615 doi 10.1111/j.1744-7348.1939.tb06990.x.
Branch M A, Coleman T F, Li Y. A Subspace, Interior, and Conjugate Gradient Method for Large-Scale Bound-Constrained Minimization Problems. SIAM Journal on Scientific Computing 1999; 21(1):1-23 doi 10.1137/s1064827595289108.
Bringhen S, Mina R, Cafro A M, Liberati A M, Spada S, Belotti A, et al. Once-weekly carfilzomib, pomalidomide, and low-dose dexamethasone for relapsed/refractory myeloma: a phase I/II study. Leukemia 2018 doi 10.1038/s41375-018-0024-1.
Burki T K. Selinexor and dexamethasone in multiple myeloma. The Lancet Oncology 2018; 19(3):e146 doi.
Chou T-C, Talalay P. Quantitative analysis of dose-effect relationships: the combined effects of multiple drugs or enzyme inhibitors. Advances in Enzyme Regulation 1984; 22:27-55 doi.
Chou T C. Theoretical basis, experimental design, and computerized simulation of synergism and antagonism in drug-combination studies (vol 58, pg 621, 2006). Pharmacol Rev 2007; 59(1):124-.
Dimopoulos M A, Lonial S, White D, Moreau P, Palumbo A, San-Miguel J, et al. Elotuzumab plus lenalidomide/dexamethasone for relapsed or refractory multiple myeloma: ELOQUENT-2 follow-up and post-hoc analyses on progression-free survival and tumour growth. Brit J Haematol 2017; 178(6):896-905 doi 10.1111/bjh.14787.
Dimopoulos M A, White D J, Benboubker L, Cook G, Leiba M, Morton J, et al. Daratumumab, Lenalidomide, and Dexamethasone (DRd) Versus Lenalidomide and Dexamethasone (Rd) in Relapsed or Refractory Multiple Myeloma (RRMM): Updated Efficacy and Safety Analysis of Pollux. Blood 2017; 130(Suppl 1):739-.
Durie B G M, Harousseau J L, Miguel J S, Blade J, Barlogie B, Anderson K, et al. International uniform response criteria for multiple myeloma. Leukemia 2006; 20(9):1467-73 doi 10.1038/sj.leu.2404284.
Durie B G M, Hoering A, Abidi M H, Rajkumar S V, Epstein J, Kahanic S P, et al. Bortezomib with lenalidomide and dexamethasone versus lenalidomide and dexamethasone alone in patients with newly diagnosed myeloma without intent for immediate autologous stem-cell transplant (SWOG S0777): a randomised, open-label, phase 3 trial. The Lancet 2017; 389(10068):519-27 doi.
Facon T, Kumar S K, Plesner T, Orlowski R Z, Moreau P, Bahlis N, et al. Phase 3 Randomized Study of Daratumumab Plus Lenalidomide and Dexamethasone (D-Rd) Versus Lenalidomide and Dexamethasone (Rd) in Patients with Newly Diagnosed Multiple Myeloma (NDMM) Ineligible for Transplant (MAIA). Blood 2018; 132(Suppl 1):LBA-2-LBA-doi 10.1182/blood-2018-120737.
FDA. 2019 30 Aug. 2019. FDA grants accelerated approval to selinexor for multiple myeloma. 30 Aug. 2019.
Foucquier J, Guedj M. Analysis of drug combinations: current methodological landscape. Pharmacol Res Perspect 2015; 3(3):e00149 doi 10.1002/prp2.149.
Fowler J F. The linear-quadratic formula and progress in fractionated radiotherapy. The British Journal of Radiology 1989; 62(740):679-94 doi 10.1259/0007-1285-62-740-679.
Griffin R J. International Journal of Radiation Oncology Biology Physics 2006; 66(2):627 doi 10.1016/j.ijrobp.2006.06.027.
Hassan Zafar M, Khan A, Aggarwal S, Bhargava M. Efficacy and tolerability of bortezomib and dexamethasone in newly diagnosed multiple myeloma. South Asian Journal of Cancer 2018; 7(1):58-60 doi 10.4103/sajc.sajc_59_17.
Khin Z P, Ribeiro M L, Jacobson T, Hazlehurst L, Perez L, Baz R, et al. A preclinical assay for chemosensitivity in multiple myeloma. Cancer Res 2014; 74(1):56-67 doi 10.1158/0008-5472.CAN-13-2397.
Kirkpatrick J P, Marks L B. Modeling killing and repopulation kinetics of subclinical cancer: direct calculations from clinical data. International Journal of Radiation Oncology*Biology*Physics 2004; 58(2):641-54 doi.
Loewe S. The problem of synergism and antagonism of combined drugs. Arzneimittelforschung 1953; 3(6):285-90.
Lopez J S, Banerji U. Combine and conquer: challenges for targeted therapy combinations in early phase trials. Nat Rev Clin Oncol 2017; 14(1):57-66 doi 10.1038/nrclinonc.2016.96.
Meyer C T, Wooten D J, Paudel B B, Bauer J, Hardeman K N, Westover D, et al. Quantifying Drug Combination Synergy along Potency and Efficacy Axes. Cell Syst 2019; 8(2):97-108.e16 doi.
Moreau P, Chanan-Khan A A, Roberts A W, Agarwal A B, Facon T, Kumar S, et al. Venetoclax Combined with Bortezomib and Dexamethasone for Patients with Relapsed/Refractory Multiple Myeloma. Blood 2016; 128(22):975-.
Moreau P, Masszi T, Grzasko N, Bahlis N J, Hansson M, Pour L, et al. Ixazomib, an Investigational Oral Proteasome Inhibitor (PI), in Combination with Lenalidomide and Dexamethasone (IRd), Significantly Extends Progression-Free Survival (PFS) for Patients (Pts) with Relapsed and/or Refractory Multiple Myeloma (RRMM): The Phase 3 Tourmaline-MM1 Study. Blood 2015; 126(23):727-.
Neel D S, Bivona T G. Resistance is futile: overcoming resistance to targeted therapies in lung adenocarcinoma. npj Precision Oncology 2017; 1(1):3 doi 10.1038/s41698-017-0007-0.
Owen L N, Steel G G. The growth and cell population kinetics of spontaneous tumours in domestic animals. Brit J Cancer 1969; 23(3):493-509 doi 10.1038/bjc.1969.62.
Palmer A C, Sorger P K. Combination Cancer Therapy Can Confer Benefit via Patient-to-Patient Variability without Drug Additivity or Synergy. Cell 2017; 171(7):1678-+doi 10.1016/j.cell.2017.11.009.
Palumbo A, Chanan-Khan A, Weisel K, Nooka A K, Masszi T, Beksac M, et al. Daratumumab, Bortezomib, and Dexamethasone for Multiple Myeloma. New England Journal of Medicine 2016; 375(8):754-66 doi 10.1056/NEJMoa1606038.
Palumbo A, Chanan-Khan A A A, Weisel K, Nooka A K, Masszi T, Beksac M, et al. Phase III randomized controlled study of daratumumab, bortezomib, and dexamethasone (DVd) versus bortezomib and dexamethasone (Vd) in patients (pts) with relapsed or refractory multiple myeloma (RRMM): CASTOR study. J Clin Oncol 2016; 34(18_suppl):LBA4-LBA doi 10.1200/JCO.2016.34.18_suppl.LBA4.
Papadopoulos K P, Siegel D S, Vesole D H, Lee P, Rosen S T, Zojwalla N, et al. Phase I Study of 30-Minute Infusion of Carfilzomib As Single Agent or in Combination With Low-Dose Dexamethasone in Patients With Relapsed and/or Refractory Multiple Myeloma. J Clin Oncol 2015; 33(7):732-9 doi 10.1200/JCO.2013.52.3522.
Rajkumar S V. Multiple myeloma: 2018 update on diagnosis, risk-stratification, and management. American Journal of Hematology 2018; 93(8):1091-110 doi 10.1002/ajh.25117.
Renovanz M, Kim E L. Intratumoral Heterogeneity, Its Contribution to Therapy Resistance and Methodological Caveats to Assessment. Frontiers in Oncology 2014; 4(142) doi 10.3389/fonc.2014.00142.
Retsky M W, Swartzendruber D E, Wardwell R H, Bame P D. Is Gompertzian or exponential kinetics a valid description of individual human cancer growth? Med Hypotheses 1990; 33(2):95-106 doi.
Richardson P G, Moreau P, Laubach J P, Maglio M E, Lonial S, San-Miguel J. Deacetylase inhibitors as a novel modality in the treatment of multiple myeloma. Pharmacological Research 2017; 117:185-91 doi.
Richardson P G, Oriol A, Beksac M, Liberati A M, Galli M, Schjesvold F, et al. Pomalidomide, bortezomib, and dexamethasone for patients with relapsed or refractory multiple myeloma previously treated with lenalidomide (OPTIMISMM): a randomised, open-label, phase 3 trial. The Lancet Oncology 2019; 20(6):781-94 doi 10.1016/S1470-2045(19)30152-4.
Riechelmann R, Girardi D. Drug interactions in cancer patients: A hidden risk? Journal of Research in Pharmacy Practice 2016; 5(2):77-8 doi 10.4103/2279-042X.179560.
Robak P, Drozdz I, Szemraj J, Robak T. Drug resistance in multiple myeloma. Cancer Treatment Reviews 2018; 70:199-208 doi.
Salmon S, Dalton W, Grogan T, Plezia P, Lehnert M, Roe D, et al. Multidrug-resistant myeloma: laboratory and clinical effects of verapamil as a chemosensitizer. Blood 1991; 78(1):44-50.
Sherbenou D W, Mark T M, Forsberg P. Monoclonal Antibodies in Multiple Myeloma: A New Wave of the Future. Clinical Lymphoma Myeloma and Leukemia 2017; 17(9):545-54 doi.
Silva A, Jacobson T, Meads M, Distler A, Shain K. An Organotypic High Throughput System for Characterization of Drug Sensitivity of Primary Multiple Myeloma Cells. Jove-J Vis Exp 2015(101) doi ARTN e53070
Silva A, Silva M C, Sudalagunta P, Distler A, Jacobson T, Collins A et al. An Ex Vivo Platform for the Prediction of Clinical Response in Multiple Myeloma. Cancer Research 2017; 77(12):3336-51 doi 10.1158/0008-5472.Can-17-0502.
Sonneveld P. Multidrug resistance in haematological malignancies. Journal of Internal Medicine 2000; 247(5):521-34 doi 10.1046/j.1365-2796.2000.00689.x.
Stewart A K, Dimopoulos M A, Masszi T, Špička I, Oriol A, Hájek R, et al. Health-Related Quality-of-Life Results From the Open-Label, Randomized, Phase III ASPIRE Trial Evaluating Carfilzomib, Lenalidomide, and Dexamethasone Versus Lenalidomide and Dexamethasone in Patients With Relapsed Multiple Myeloma. Journal of clinical oncology: official journal of the American Society of Clinical Oncology 2016; 34(32):3921-30 doi 10.1200/JCO.2016.66.9648.
Wood K, Nishida S, Sontag E D, Cluzel P. Mechanism-independent method for predicting response to multidrug combinations in bacteria. P Natl Acad Sci USA 2012; 109(30):12254-9 doi 10.1073/pnas.1201281109.
Zierhut M L, Yen Y-F, Chen A P, Bok R, Albers M J, Zhang V, et al. Kinetic modeling of hyperpolarized 13C1-pyruvate metabolism in normal rats and TRAMP mice. Journal of Magnetic Resonance 2010; 202(1):85-92 doi.
Zimmer A, Katzir I, Dekel E, Mayo A E, Alon U. Prediction of multidimensional drug dose responses based on measurements of drug pairs. Proceedings of the National Academy of Sciences 2016; 113(37):10442-7 doi 10.1073/pnas.1606301113.

1. A method detecting synergistic drug combinations for the treatment of a cancer comprising
(a) culturing a plurality of cells from a subject in a chamber;
(b) contacting the cells in the chamber with a first active agent;
(c) measuring the concentration of the first active agent at a first time point;
(d) capturing a first optical signal from the cells contacted with the first active agent at a first time point;
(e) measuring the concentration of the first active agent at a second time point;
(f) capturing a second optical signal from the cells contacted with the first active agent at a second time point;
(g) analyzing the first optical signal and the second optical signal to detect cell membrane motion of the cells;
(h) analyzing the cell membrane motion to quantify the viability of the cells following contact with the first active agent thereby detecting the drug induced damage at the second time point;
(i) measuring the repair rate of the cells, therapeutic threshold, rate of sensitivity of therapy, and/or clonal composition of the tumor;
(j) repeating steps (a)-(i) with a second active agent; and
(k) calculating the synergistic effect of each active agent or pair of active agents using an ex vivo mathematical malignancy advisor (EMMA) comprising a synergy augmented model (SAM).

2. The method of claim 1, wherein the first optical signal, the second optical signal, or a combination thereof comprises an image.

3. The method of claim 1, wherein the first optical signal, the second optical signal, or a combination thereof comprises a bright field image.

4. The method of claim 1, wherein the absence of cell membrane motion indicates cell death.

5. The method of claim 1, further comprising repeating steps (a)-(i) using the first and second active agents in combination.

6. The method of claim 1, wherein the cells comprise cancer.

7. The method claim 6, further comprising selecting a cancer treatment regimen for the subject based on the results of the ex vivo mathematical malignancy advisor (EMMA) comprising a synergy augmented model (SAM).

8. The method of claim 6, wherein the cancer comprises a hematological cancer.

9. The method of claim 6, wherein the cancer comprises multiple myeloma.

10. The method of claim 6, wherein chamber recapitulates the cancer microenvironment.

11. The method of claim 10, wherein the chamber comprises extracellular matrix, subject-derived stroma, and growth factors to recapitulate the cancer microenvironment.

12. The method of claim 1, wherein the chamber comprises a microfluidic chamber.

13. The method of claim 1, wherein the chamber comprises a well in a multi-well plate.
